SEQUENCE LISTING (19 sequences)

SEQ ID NO: 1 - protein, 1014 aa, Homo sapiens
  Poly (ADP-ribose) polymerase 1
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 2 - protein, 1014 aa, Artificial Sequence
  Poly (ADP-ribose) polymerase 1 E988Q
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNQYIVYDIAQVNLKYLLKLKFNFKTSLW

SEQ ID NO: 3 - protein, 1013 aa, Mus musculus
  Poly (ADP-ribose) polymerase 1, isoform 1
MAEASERLYRVQYAKSGRASCKKCSESIPKDSLRMAIMVQSPMFDGKVPHWYHFSCFWKVGQSIRHPDVEVDGFSELRWDDQQKVKKTAEAGGVAGKGQDGSGGKAEKTLGDFAAEYAKSNRSMCKGCLEKIEKGQMRLSKKMVDPEKPQLGMIDRWYHPTCFVKKRDELGFRPEYSASQLKGFSLLSAEDKEALKKQLPAIKNEGKRKGDEVDGTDEVAKKKSRKETDKYSKLEKALKAQNELIWNIKDELKKACSTNDLKELLIFNQQQVPSGESAILDRVADGMAFGALLPCKECSGQLVFKSDAYYCTGDVTAWTKCMVKTQNPSRKEWVTPKEFREISYLKKLKVKKQDRIFPPESSAPITVHWPLSVTSAPTAVNSSAPADKPLSNMKILTLGKLSQNKDEAKAVIEKLGGKLTGSANKASLCISIKKEVEKMNKKMEEVKEANIRVVSEDFLQDVSASTKSLQDLLSAHSLSPWGAEVKAEPGEVVAPRGKSAAPSKKSKGCFKEEGVNKSEKRMKLTLKGGAAVDPDSGLEHSAHVLEKGGKVFSATLGLVDIVKGTNSYYKLQLLEDDKESRYWIFRSWGRLGTVIGSNKLEQMPSKEEAVEQFMKLYEEKTGNAWHSKNFTKYPKKFYPLEIDYGQDEEAVKKLTVKPGTKSKLPKPVQELVGMIFDVDSMKKALVEYEIDLQKMPLGKLSRRQIQAAYSILSEVQQPVSQGSSESQILDLSNRFYTLIPHDFGMKKPPLLNNADSVQAKVEMLDNLLDIEVAYSLLRGGSDDSSKDPIDVNYEKLKTDIKVVDRDSEEAEVIRKYVKNTHATTHNAYDLEVIDIFKIEREGESQRYKPFRQLHNRRLLWHGSRTTNFAGILSQGLRIAPPEAPVTGYMFGKGIYFADMVSKSANYCHTSQGDPIGLIMLGEVALGNMYELKHASHISKLPKGKHSVKGLGKTTPDPSASITLEGVEVPLGTGIPSGVNDTALLYNEYIVYDIAQVNLKYLLKLKFNFKTSLW

SEQ ID NO: 4 - protein, 492 aa, Mus musculus
  Poly (ADP-ribose) polymerase 1, isoform 2
MKLTLKGGAAVDPDSGLEHSAHVLEKGGKVFSATLGLVDIVKGTNSYYKLQLLEDDKESRYWIFRSWGRLGTVIGSNKLEQMPSKEEAVEQFMKLYEEKTGNAWHSKNFTKYPKKFYPLEIDYGQDEEAVKKLTVKPGTKSKLPKPVQELVGMIFDVDSMKKALVEYEIDLQKMPLGKLSRRQIQAAYSILSEVQQPVSQGSSESQILDLSNRFYTLIPHDFGMKKPPLLNNADSVQAKVEMLDNLLDIEVAYSLLRGGSDDSSKDPIDVNYEKLKTDIKVVDRDSEEAEVIRKYVKNTHATTHNAYDLEVIDIFKIEREGESQRYKPFRQLHNRRLLWHGSRTTNFAGILSQGLRIAPPEAPVTGYMFGKGIYFADMVSKSANYCHTSQGDPIGLIMLGEVALGNMYELKHASHISKLPKGKHSVKGLGKTTPDPSASITLEGVEVPLGTGIPSGVNDTALLYNEYIVYDIAQVNLKYLLKLKFNFKTSLW

SEQ ID NO: 5 - protein, 1014 aa, Rattus norvegicus
  Poly (ADP-ribose) polymerase 1
MAEATERLYRVEYAKSGRASCKKCSESIPKDSLRMAIMVQSPMFDGKVPHWYHFSCFWKVGHSIRQPDTEVDGFSELRWDDQQKVKKTAEAGGVAGKGQHGGGGKAEKTLGDFAAEYAKSNRSTCKGCMEKIEKGQMRLSKKMLDPEKPQLGMIDRWYHPTCFVKNRDELGFRPEYSASQLKGFSLLSAEDKEALKKQLPAVKSEGKRKCDEVDGIDEVAKKKSKKGKDKESSKLEKALKAQNELVWNIKDELKKACSTNDLKELLIFNQQQVPSGESAILDRVADGMAFGALLPCKECSGQLVFKSDAYYCTGDVTAWTKCMVKTQNPSRKEWVTPKEFREISYLKKLKIKKQDRLFPPESSAPAPPAPPVSITSAPTAVNSSAPADKPLSNMKILTLGKLSQNKDEAKAMIEKLGGKLTGSANKASLCISTKKEVEKMSKKMEEVKAANVRVVCEDFLQDVSASAKSLQELLSAHSLSSWGAEVKVEPGEVVVPKGKSAAPSKKSKGAVKEEGVNKSEKRMKLTLKGGAAVDPDSGLEHSAHVLEKGGKVFSATLGLVDIVKGTNSYYKLQLLESDKESRYWIFRSWGRVGTVIGSNKLEQMPSKEDAVEHFMKLYEEKTGNAWHSKNFTKYPKKFYPLEIDYGQDEEAVKKLAVKPGTKSKLPKPVQELVGMIFDVESMKKALVEYEIDLQKMPLGKLSRRQIQAAYSILSEVQQAVSQGSSESQILDLSNRFYTLIPHDFGMKKPPLLNNTDSVQAKVEMLDNLLDIEVAYSLLRGGSDDSSKDPIDVNYEKLKTDIKVVDRDSEEAEVIRKYVKNTHATTHNAYDLEVIDIFKIEREGESQRYKPFRQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTAPDPSASITLDGVEVPLGTGIPSGVNDTCLLYNEYIVYDIAQVNLKYLLKLKFNFKTSLW

SEQ ID NO: 6 - protein, 583 aa, Homo sapiens
  Poly (ADP-ribose) polymerase 2, isoform 1
MAARRRRSTGGGRARALNESKRVNNGNTAPEDSSPAKKTRRCQRQESKKMPVAGGKANKDRTEDKQDGMPGRSWASKRVSESVKALLLKGKAPVDPECTAKVGKAHVYCEGNDVYDVMLNQTNLQFNNNKYYLIQLLEDDAQRNFSVWMRWGRVGKMGQHSLVACSGNLNKAKEIFQKKFLDKTKNNWEDREKFEKVPGKYDMLQMDYATNTQDEEETKKEESLKSPLKPESQLDLRVQELIKLICNVQAMEEMMMEMKYNTKKAPLGKLTVAQIKAGYQSLKKIEDCIRAGQHGRALMEACNEFYTRIPHDFGLRTPPLIRTQKELSEKIQLLEALGDIEIAIKLVKTELQSPEHPLDQHYRNLHCALRPLDHESYEFKVISQYLQSTHAPTHSDYTMTLLDLFEVEKDGEKEAFREDLHNRMLLWHGSRMSNWVGILSHGLRIAPPEAPITGYMFGKGIYFADMSSKSANYCFASRLKNTGLLLLSEVALGQCNELLEANPKAEGLLQGKHSTKGLGKMAPSSAHFVTLNGSTVPLGPASDTGILNPDGYTLNYNEYIVYNPNQVRMRYLLKVQFNFLQLW

SEQ ID NO: 7 - protein, 570 aa, Homo sapiens
  Poly (ADP-ribose) polymerase 2, isoform 2
MAARRRRSTGGGRARALNESKRVNNGNTAPEDSSPAKKTRRCQRQESKKMPVAGGKANKDRTEDKQDESVKALLLKGKAPVDPECTAKVGKAHVYCEGNDVYDVMLNQTNLQFNNNKYYLIQLLEDDAQRNFSVWMRWGRVGKMGQHSLVACSGNLNKAKEIFQKKFLDKTKNNWEDREKFEKVPGKYDMLQMDYATNTQDEEETKKEESLKSPLKPESQLDLRVQELIKLICNVQAMEEMMMEMKYNTKKAPLGKLTVAQIKAGYQSLKKIEDCIRAGQHGRALMEACNEFYTRIPHDFGLRTPPLIRTQKELSEKIQLLEALGDIEIAIKLVKTELQSPEHPLDQHYRNLHCALRPLDHESYEFKVISQYLQSTHAPTHSDYTMTLLDLFEVEKDGEKEAFREDLHNRMLLWHGSRMSNWVGILSHGLRIAPPEAPITGYMFGKGIYFADMSSKSANYCFASRLKNTGLLLLSEVALGQCNELLEANPKAEGLLQGKHSTKGLGKMAPSSAHFVTLNGSTVPLGPASDTGILNPDGYTLNYNEYIVYNPNQVRMRYLLKVQFNFLQLW

SEQ ID NO: 8 - protein, 559 aa, Mus musculus
  Poly (ADP-ribose) polymerase 2
MAPRRQRSGSGRRVLNEAKKVDNGNKATEDDSPPGKKMRTCQRKGPMAGGKDADRTKDNRDSVKTLLLKGKAPVDPECAAKLGKAHVYCEGDDVYDVMLNQTNLQFNNNKYYLIQLLEDDAQRNFSVWMRWGRVGKTGQHSLVTCSGDLNKAKEIFQKKFLDKTKNNWEDRENFEKVPGKYDMLQMDYAASTQDESKTKEEETLKPESQLDLRVQELLKLICNVQTMEEMMIEMKYDTKRAPLGKLTVAQIKAGYQSLKKIEDCIRAGQHGRALVEACNEFYTRIPHDFGLSIPPVIRTEKELSDKVKLLEALGDIEIALKLVKSERQGLEHPLDQHYRNLHCALRPLDHESNEFKVISQYLQSTHAPTHKDYTMTLLDVFEVEKEGEKEAFREDLPNRMLLWHGSRLSNWVGILSHGLRVAPPEAPITGYMFGKGIYFADMSSKSANYCFASRLKNTGLLLLSEVALGQCNELLEANPKAQGLLRGKHSTKGMGKMAPSPAHFITLNGSTVPLGPASDTGILNPEGYTLNYNEFIVYSPNQVRMRYLLKIQFNFLQLW

SEQ ID NO: 9 - protein, 346 aa, Homo sapiens [sequence omitted]

SEQ ID NO: 10 - protein, 346 aa, Mus musculus
  Histone PARylation factor 1
MVGGGGKRRTAGAGPQCEKTVEVKKSKFSEADVSSDLRKEVENLYKLSLPEDFYHFWKFCEELDPEKPADALATSLGLRLVGPYDILAGKHKMKKKPTGLNCNLHWRFYYDPPEFQTIIIGDNKTQYHMGYFRDSPDELPVYVGTNEAKKNCIIIQNGDNVFAAIKLFLMKKLKEVTDRKKISILKNIDEKLTEAARKLGYSLEQRTVKMRQRDKKVVTKTFHGAGLVVPVDKNDVGYRELPETDADLKRICKAVVDAASDEERLKAFAPIQEMMTFVQFANDECDYGMGLELGMDLFCYGSHYFHKVAGQLLPLAYNLLKRDLFAKIIEDHLASRSEENIDQLAG

SEQ ID NO: 11 - protein, 976 aa, Homo sapiens
  Poly (ADP-ribose) glycohydrolase, isoform 1
MNAGPGCEPCTKRPRWGAATTSPAASDARSFPSRQRRVLDPKDAHVQFRVPPSSPACVPGRAGQHRGSATSLVFKQKTITSWMDTKGIKTAESESLDSKENNNTRIESMMSSVQKDNFYQHNVEKLENVSQLSLDKSPTEKSTQYLNQHQTAAMCKWQNEGKHTEQLLESEPQTVTLVPEQFSNANIDRSPQNDDHSDTDSEENRDNQQFLTTVKLANAKQTTEDEQAREAKSHQKCSKSCDPGEDCASCQQDEIDVVPESPLSDVGSEDVGTGPKNDNKLTRQESCLGNSPPFEKESEPESPMDVDNSKNSCQDSEADEETSPGFDEQEDGSSSQTANKPSRFQARDADIEFRKRYSTKGGEVRLHFQFEGGESRTGMNDLNAKLPGNISSLNVECRNSKQHGKKDSKITDHFMRLPKAEDRRKEQWETKHQRTERKIPKYVPPHLSPDKKWLGTPIEEMRRMPRCGIRLPLLRPSANHTVTIRVDLLRAGEVPKPFPTHYKDLWDNKHVKMPCSEQNLYPVEDENGERTAGSRWELIQTALLNKFTRPQNLKDAILKYNVAYSKKWDFTALIDFWDKVLEEAEAQHLYQSILPDMVKIALCLPNICTQPIPLLKQKMNHSITMSQEQIASLLANAFFCTFPRRNAKMKSEYSSYPDINFNRLFEGRSSRKPEKLKTLFCYFRRVTEKKPTGLVTFTRQSLEDFPEWERCEKPLTRLHVTYEGTIEENGQGMLQVDFANRFVGGGVTSAGLVQEEIRFLINPELIISRLFTEVLDHNECLIITGTEQYSEYTGYAETYRWSRSHEDGSERDDWQRRCTEIVAIDALHFRRYLDQFVPEKMRRELNKAYCGFLRPGVSSENLSAVATGNWGCGAFGGDARLKALIQILAAAAAERDVVYFTFGDSELMRDIYSMHIFLTERKLTVGDVYKLLLRYYNEECRNCSTPGPDIKLYPFIYHAVESCAETADHSGQRTGT

SEQ ID NO: 12 - protein, 894 aa, Homo sapiens
  Poly (ADP-ribose) polymerase 1, isoform 2
MDTKGIKTAESESLDSKENNNTRIESMMSSVQKDNFYQHNVEKLENVSQLSLDKSPTEKSTQYLNQHQTAAMCKWQNEGKHTEQLLESEPQTVTLVPEQFSNANIDRSPQNDDHSDTDSEENRDNQQFLTTVKLANAKQTTEDEQAREAKSHQKCSKSCDPGEDCASCQQDEIDVVPESPLSDVGSEDVGTGPKNDNKLTRQESCLGNSPPFEKESEPESPMDVDNSKNSCQDSEADEETSPGFDEQEDGSSSQTANKPSRFQARDADIEFRKRYSTKGGEVRLHFQFEGGESRTGMNDLNAKLPGNISSLNVECRNSKQHGKKDSKITDHFMRLPKAEDRRKEQWETKHQRTERKIPKYVPPHLSPDKKWLGTPIEEMRRMPRCGIRLPLLRPSANHTVTIRVDLLRAGEVPKPFPTHYKDLWDNKHVKMPCSEQNLYPVEDENGERTAGSRWELIQTALLNKFTRPQNLKDAILKYNVAYSKKWDFTALIDFWDKVLEEAEAQHLYQSILPDMVKIALCLPNICTQPIPLLKQKMNHSITMSQEQIASLLANAFFCTFPRRNAKMKSEYSSYPDINFNRLFEGRSSRKPEKLKTLFCYFRRVTEKKPTGLVTFTRQSLEDFPEWERCEKPLTRLHVTYEGTIEENGQGMLQVDFANRFVGGGVTSAGLVQEEIRFLINPELIISRLFTEVLDHNECLIITGTEQYSEYTGYAETYRWSRSHEDGSERDDWQRRCTEIVAIDALHFRRYLDQFVPEKMRRELNKAYCGFLRPGVSSENLSAVATGNWGCGAFGGDARLKALIQILAAAAAERDVVYFTFGDSELMRDIYSMHIFLTERKLTVGDVYKLLLRYYNEECRNCSTPGPDIKLYPFIYHAVESCAETADHSGQRTGT

SEQ ID NO: 13 - protein, 868 aa, Homo sapiens
  Poly (ADP-ribose) glycohydrolase, isoform 3
MMSSVQKDNFYQHNVEKLENVSQLSLDKSPTEKSTQYLNQHQTAAMCKWQNEGKHTEQLLESEPQTVTLVPEQFSNANIDRSPQNDDHSDTDSEENRDNQQFLTTVKLANAKQTTEDEQAREAKSHQKCSKSCDPGEDCASCQQDEIDVVPESPLSDVGSEDVGTGPKNDNKLTRQESCLGNSPPFEKESEPESPMDVDNSKNSCQDSEADEETSPGFDEQEDGSSSQTANKPSRFQARDADIEFRKRYSTKGGEVRLHFQFEGGESRTGMNDLNAKLPGNISSLNVECRNSKQHGKKDSKITDHFMRLPKAEDRRKEQWETKHQRTERKIPKYVPPHLSPDKKWLGTPIEEMRRMPRCGIRLPLLRPSANHTVTIRVDLLRAGEVPKPFPTHYKDLWDNKHVKMPCSEQNLYPVEDENGERTAGSRWELIQTALLNKFTRPQNLKDAILKYNVAYSKKWDFTALIDFWDKVLEEAEAQHLYQSILPDMVKIALCLPNICTQPIPLLKQKMNHSITMSQEQIASLLANAFFCTFPRRNAKMKSEYSSYPDINFNRLFEGRSSRKPEKLKTLFCYFRRVTEKKPTGLVTFTRQSLEDFPEWERCEKPLTRLHVTYEGTIEENGQGMLQVDFANRFVGGGVTSAGLVQEEIRFLINPELIISRLFTEVLDHNECLIITGTEQYSEYTGYAETYRWSRSHEDGSERDDWQRRCTEIVAIDALHFRRYLDQFVPEKMRRELNKAYCGFLRPGVSSENLSAVATGNWGCGAFGGDARLKALIQILAAAAAERDVVYFTFGDSELMRDIYSMHIFLTERKLTVGDVYKLLLRYYNEECRNCSTPGPDIKLYPFIYHAVESCAETADHSGQRTGT

SEQ ID NO: 14 - protein, 969 aa, Mus musculus
  Poly (ADP-ribose) glycohydrolase, isoform 1
MSAGPGWEPCTKRPRWGAAGTSAPTASDSRSFPGRQRRVLDPKDAPVQFRVPPSSPACVSGRAGPHRGNATSFVFKQKTITTWMDTKGPKTAESESKENNNTRIDSMMSSVQKDNFYPHKVEKLENVPQLNLDKSPTEKSSQYLNQQQTASVCKWQNEGKHAEQLLASEPPAGTPLPKQLSNANIGQSPHTDDHSDTDHEEDRDNQQFLTPIKLANTKPTVGDGQARSNCKCSGSRQSVKDCTGCQQEEVDVLPESPLSDVGAEDIGTGPKNDNKLTGQESSLGDSPPFEKESEPESPMDVDNSKNSCQDSEADEETSPVFDEQDDRSSQTANKLSSCQAREADGDLRKRYLTKGSEVRLHFQFEGENNAGTSDLNAKPSGNSSSLNVECRSSKQHGKRDSKITDHFMRISKSEDRRKEQCEVRHQRTERKIPKYIPPNLPPEKKWLGTPIEEMRKMPRCGIHLPSLRPSASHTVTVRVDLLRAGEVPKPFPTHYKDLWDNKHVKMPCSEQNLYPVEDENGERTAGSRWELIQTALLNKFTRPQNLKDAILKYNVAYSKKWDFTALVDFWDKVLEEAEAQHLYQSILPDMVKIALCLPNICTQPIPLLKQKMNHSVTMSQEQIASLLANAFFCTFPRRNAKMKSEYSSYPDINFNRLFEGRSSRKPEKLKTLFCYFRRVTEKKPTGLVTFTRQSLEDFPEWERCEKPLTRLHVTYEGTIEGNGRGMLQVDFANRFVGGGVTGAGLVQEEIRFLINPELIVSRLFTEVLDHNECLIITGTEQYSEYTGYAETYRWARSHEDGSEKDDWQRRCTEIVAIDALHFRRYLDQFVPEKVRRELNKAYCGFLRPGVPSENLSAVATGNWGCGAFGGDARLKALIQILAAAAAERDVVYFTFGDSELMRDIYSMHTFLTERKLDVGKVYKLLLRYYNEECRNCSTPGPDIKLYPFIYHAVESSAETTDMPGQKAGT

SEQ ID NO: 15 - protein, 920 aa, Mus musculus
  Poly (ADP-ribose) glycohydrolase, isoform 2
MSAGPGWEPCTKRPRWGAAGTSAPTASDSRSFPGRQRRVLDPKDAPVQFRVPPSSPACVSGRAGPHRGNATSFVFKQKTITTWMDTKGPKTAESESKENNNTRIDSMMSSVQKDNFYPHKVEKLENVPQLNLDKSPTEKSSQYLNQQQTASVCKWQNEGKHAEQLLASEPPAGTPLPKQLSNANIGQSPHTDDHSDTDHEEDRDNQQFLTPIKLANTKPTVGDGQARSNCKCSGSRQSVKDCTGCQQEEVDVLPESPLSDVGAEDIGTGPKNDNKLTGQESSLGDSPPFEKESEPESPMDVDNSKNSCQDSEADEETSPVFDEQDDRSSQTANKLSSCQAREADGDLRKRYLTKGSEVRLHFQFEGENNAGTSDLNAKPSGNSSSLNVECRSSKQHGKRDSKITDHFMRISKSEDRRKEQCEVRHQRTERKIPKYIPPNLPPEKKWLGTPIEEMRKMPRCGIHLPSLRPSASHTVTVRVDLLRAGEVPKPFPTHYKDLWDNKHVKMPCSEQNLYPVEDENGERTAGSRWELIQTALLNKFTRPQNLKDAILKYNVAYSKKWDFTALVDFWDKVLEEAEAQHLYQSILPDMVKIALCLPNICTQPIPLLKQKMNHSVTMSQEQIASLLANAFFCTFPRRNAKMKSEYSSYPDINFNRLFEGRSSRKPEKLKTLFCYFRRVTEKKPTGLVTFTRQSLEDFPEWERCEKPLTRLHVTYEGTIEGNGRGMLQVDFANRFVGGGVTGAGLVQEEIRFLINPELIVSRLFTEVLDHNECLIITGTEQYSEYTGYAETYRWARSHEDGSEKDDWQRRCTEIVAIDALHFRRYLDQFVPEKVRRELNKAYCGFLRPGVPSENLSAVATGNWGCGAFGGDARLKALIQILAAAAAERDVVYFTFGDSELMRDIYSMHTFLTERKLDVGE

SEQ ID NO: 16 - protein, 972 aa, Rattus norvegicus
  Poly (ADP-ribose) glycohydrolase
MSAGPGCEPCTKRPRWGAAGTSAPTASDSRSFPGRQKRVLDPKDAPVQFRVPPSSSACVSGRAGPHRGSVTSFVFKQKPITTWMDTKGPKTAESESKENNNTRTDPMMSSVQKDNFYPHKVEKLGNVPQLNLDKSPTEKSTPYLNQQQTAGVCKWHSAGERAEQLSASEPSAVTQAPKQLSNANIDQSPPTDGHSDTDHEEDRDNQQFLTPVKLANAKQTVGDGQARSNCKCSASCQCGQDCAGCQREEADVIPESPLSDVGAEDIGTGSKNDNKLTGQESGLGDSPPFEKESEPESPMDVDNSKTSCQDSEADEEASPVFDEQDDQDDRSSQTANKLSSRQAREVDGDLRKRYLTKGSEIRLHFQFEGGSNAGTSDLNAKPSGNSSSLNVDGRSSKQHGKRDSKITDHFVRIPKSEDKRKEQCEVRHQRAERKIPKYVPPNLPPDKKWLGTPIEEMRKMPRCGVRLPLLRPSASHTVTVRVDLLRAGEVPKPFPTHYKDLWDNKHVKMPCSEQNLYPVEDENGERTAGSRWELIQTALLNKFTRPQNLKDAILKYNVAYSKKWDFTALVDFWDKVLEEAEAQHLYQSILPDMVKIALCLPNICTQPIPLLKQKMNHSVTMSQEQIASLLANAFFCTFPRRNAKMKSEYSSYPDINFNRLFEGRSSRKPEKLKTLFCYFRRVTEKKPTGLVTFTRQSLEDFPEWERCDKPLTRLHVTYEGTIEGNGRGMLQVDFANRFVGGGVTGAGLVQEEIRFLINPELIVSRLFTEVLDHNECLIITGTEQYSEYTGYAETYRWARSHEDGSEKDDWQRCCTEIVAIDALHFRRYLDQFVPEKVRRELNKAYCGFLRPGVPPENLSAVATGNWGCGAFGGDARLKALIQLLAAAAAERDVVYFTFGDSELMRDIYSMHTFLTERKLNVGKVYRLLLRYYREECRDCSSPGPDTKLYPFIYHAAESSAETSDQPGQRTGT

SEQ ID NO: 17 - protein, 363 aa, Homo sapiens
  Poly (ADP-ribose) glycohydrolase ARH3
MAAAAMAAAAGGGAGAARSLSRFRGCLAGALLGDCVGSFYEAHDTVDLTSVLRHVQSLEPDPGTPGSERTEALYYTDDTAMARALVQSLLAKEAFDEVDMAHRFAQEYKKDPDRGYGAGVVTVFKKLLNPKCRDVFEPARAQFNGKGSYGNGGAMRVAGISLAYSSVQDVQKFARLSAQLTHASSLGYNGAILQALAVHLALQGESSSEHFLKQLLGHMEDLEGDAQSVLDARELGMEERPYSSRLKKIGELLDQASVTREEVVSELGNGIAAFESVPTAIYCFLRCMEPDPEIPSAFNSLQRTLIYSISLGGDTDTIATMAGAIAGAYYGMDQVPESWQQSCEGYEETDILAQSLHRVFQKS

SEQ ID NO: 18 - protein, 370 aa, Mus musculus
  Poly (ADP-ribose) glycohydrolase ARH3, isoform 1
MAVAAAAAATAMSAAGGGGASAARSISRFRGCLAGALLGDCVGAVYEAHDTVSLASVLSHVESLEPDPGTPGSARTETLYYTDDTAMTRALVQSLLAKEAFDEVDMAHRFAQEYKKDPDRGYGAGVITVFKKLLNPKCRDVYEPARAQFNGKGSYGNGGAMRVAGISLAYSSVQDVQKFARLSAQLTHASSLGYNGAILQALAVHLALQGVSSSEHFLEQLLGHMEELEGDAQSVLDAKELGMEERPYSSRLKKVGELLDQDVVSREEVVSELGNGIAAFESVPTAIYCFLRCMEPHPEIPSTFNSLQRTLIYSISLGGDTDTIATMAGAIAGAYYGMEQVPESWQQSCEGFEETDVLAQSLHRVFQESS

SEQ ID NO: 19 - protein, 284 aa, Mus musculus
  Poly (ADP-ribose) glycohydrolase ARH3, isoform 2
MTRALVQSLLAKEAFDEVDMAHRFAQEYKKDPDRGYGAGVITVFKKLLNPKCRDVYEPARAQFNGKGSYGNGGAMRVAGISLAYSSVQDVQKFARLSAQLTHASSLGYNGAILQALAVHLALQGVSSSEHFLEQLLGHMEELEGDAQSVLDAKELGMEERPYSSRLKKVGELLDQDVVSREEVVSELGNGIAAFESVPTAIYCFLRCMEPHPEIPSTFNSLQRTLIYSISLGGDTDTIATMAGAIAGAYYGMEQVPESWQQSCEGFEETDVLAQSLHRVFQESS

(19) United States
(12) Patent Application Publication
(10) Pub. No.: US 2021/0363176 A1
(43) Pub. Date: Nov. 25, 2021

(54) MEANS AND METHODS FOR PRODUCTION OF SERINE ADP-RIBOSYLATED FORMS OF PROTEINS AND PEPTIDES

(71) Applicant: MAX-PLANCK-GESELLSCHAFT ZUR FÖRDERUNG DER WISSENSCHAFTEN E.V., Munich (DE)

(72) Inventors: Juan José BONFIGLIO, Munich (DE); Thomas COLBY, Köln (Cologne) (DE); Ivan MATIC, Köln (Cologne) (DE)

(21) Appl. No.: 16/757,094

(22) PCT Filed: Oct. 18, 2018
(86) PCT No.: PCT/EP2018/078592

§ 371 Date: Apr. 17, 2020

(30) Foreign Application Priority Data
Oct. 20, 2017 (EP) 17197550.1
Jan. 31, 2018 (EP) 18154508.8

(51) Int. Cl. C07K 1/02

(52) U.S. Cl. CPC C07K 1/02

(57) ABSTRACT

The present invention relates to a method for the production of a serine ADP-ribosylated protein or peptide comprising preparing an aqueous buffered solution comprising 5 to 60 mM, preferably 10 to 60 mM of a buffer and having a pH between 5.0 and 9.0, preferably between 5.5 to 8.5, and most preferably between 6.1 to 8.3, said solution further comprising (a) 0.2 to 2.5 mM NAD⁺, (b) at least 50 nM, preferably 50 to 3000 nM PARP-1, PARP-2 or the PARP-1 variant E988Q, (c) at least 100 nM, preferably 100 to 5000 nM HPF1, (d) at least 10 μg/mL, preferably 10 μg/mL to 200 μg/mL sonicated DNA, said sonicated DNA preferably comprising DNA fragments of 10 to 330 bp, and (e) up to 600 μM protein or peptide, said protein or peptide comprising at least one serine, thereby generating a reaction mix, in which the protein or peptide becomes serine ADP-ribosylated.

### RELATED PATENT APPLICATION

This patent application is a 35 U.S.C. 371 national phase patent application of International Application No. PCT/EP2018/078592, filed on Oct. 18, 2018, entitled “MEANS AND METHODS FOR PRODUCTION OF SERINE ADP-RIBOSYLATED FORMS OF PROTEINS AND PEPTIDES,” naming Juan Jose Bonfiglio et al. as inventors, and designated by attorney docket no. AA1836 PCT, which claims priority to European Application No. 18154508.8 filed on Jan. 31, 2018, and European Application No. 17197550.1, filed on Oct. 20, 2017. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy is named Sequence_Listing and is 114 kilobytes in size.

The present invention relates to a method for the production of a serine ADP-ribosylated protein or peptide comprising preparing an aqueous buffered solution comprising 5 to 60 mM, preferably 10 to 60 mM of a buffer and having a pH between 5.0 and 9.0, preferably between 5.5 to 8.5, and most preferably between 6.1 to 8.3, said solution further comprising (a) 0.2 to 2.5 mM NAD⁺, (b) at least 50 nM, preferably 50 to 3000 nM PARP-1, PARP-2 or the PARP-1 variant E988Q, (c) at least 100 nM, preferably 100 to 5000 nM HPF1, (d) at least 10 μg/mL, preferably 10 μg/mL to 200 μg/mL sonicated DNA, said sonicated DNA preferably comprising DNA fragments of 10 to 330 bp, and (e) up to 600 μM protein or peptide, said protein or peptide comprising at least one serine, thereby generating a reaction mix, in which the protein or peptide becomes serine ADP-ribosylated.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The present invention is in the technical field of a chemically complex protein post-translational modification (PTM) called ADP-ribosylation (ADPr). The biological and clinical importance of this modification is well established. This PTM and the enzymes responsible for its synthesis (known as PARPs) have been found to play a role in many key cellular processes, including maintenance of genomic stability, cell differentiation and proliferation, cytoplasmic stress responses, and microbial virulence. Moreover, dysregulation of ADPr has been linked to diseases including cancer, diabetes, neurodegenerative disorders, and heart failure, leading to the development of therapeutic PARP inhibitors, many of which are currently in clinical trials. In particular, the PARP-1 inhibitors Olaparib (tradename Lynparza) and Rucaparib (trade name Rubraca) are EU- and FDA-approved therapeutic agents for cancer therapy.

Despite the clear biological and clinical importance of ADPr, further progress in the field is limited by the difficulties in elucidating the underlying molecular mechanisms. This is due to lack of essential tools and reagents, ranging from the modified peptides themselves up to site-specific antibodies, tools which are commercially available for other biologically important protein modifications.

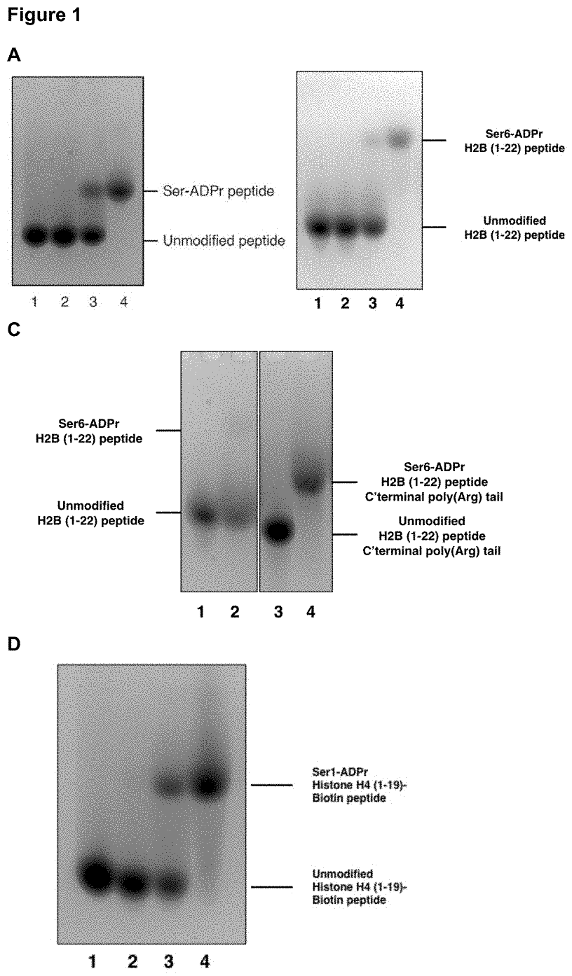

Recently the inventors reported that ADPr can be attached to serine, a previously unknown and unexpected amino acid target residue for this PTM (Leidecker et al., 2016, Nat Chem Biol 12, 998-1000). Shortly afterwards it was shown that this new form of ADPr, which is called serine-ADP-ribosylation (Ser-ADPr), is a widespread PTM that targets hundreds of different substrates, including PARP-1 itself (Bonfiglio et al., 2017, Mol Cell 65, 932-940 e936). The required elements for the in vitro modification of a target substrate with ADP-ribose on serine are PARP-1 or -2, HPF1, NAD⁺, activated (i.e. sonicated) DNA and the substrate itself, all of them contained in a reactor (e.g. a tube) in which an uncontrolled ADP-ribosylation of both the substrate and the modifying enzyme occurs (Bonfiglio et al., 2017, Mol Cell 65, 932-940 e936). While the inventors have already published serine ADP-ribosylation of particular peptides and proteins (Bonfiglio et al., 2017, Mol Cell 65, 932-940 e936), the methodology reported in the state of the art does not produce pure serine ADP-ribosylated proteins or peptides. In particular, as revealed in the appended Example 1 of the present application, the in vitro modification of 500 μM of a synthetic peptide under the experimental conditions published in the state of the art results in an incomplete modification of the substrate (FIG. 1A). Similarly, for some substrate peptides, the reaction performed according to this public methodology is very inefficient (FIGS. 1B and 1D).

Hence, the technical problem of the present invention is the provision of a method resulting in essentially pure serine ADP-ribosylated forms of proteins or peptides, in particular in a cost-effective manner on a scale varying from small (a few μg) to large (several milligrams).

The solution to this technical problem is achieved by providing the embodiments characterized in the claims.

Accordingly, the present invention relates in a first aspect to a method for the production of a serine ADP-ribosylated protein or peptide comprising preparing an aqueous buffered solution comprising 5 to 60 mM, preferably 10 to 60 mM of a buffer and having a pH between 5.0 and 9.0, preferably between 5.5 to 8.5, and most preferably between 6.1 to 8.3, said solution further comprising (a) 0.2 to 2.5 mM NAD⁺, (b) at least 50 nM, preferably 50 to 3000 nM PARP-1, PARP-2 or the PARP-1 variant E988Q, (c) at least 100 nM, preferably 100 to 5000 nM HPF1, (d) at least 10 μg/mL, preferably 10 μg/mL to 200 μg/mL sonicated DNA, said sonicated DNA preferably comprising DNA fragments of 10 to 330 bp, and (e) up to 600 μM protein or peptide, said protein or peptide comprising at least one serine, thereby generating a reaction mix, in which the protein or peptide becomes serine ADP-ribosylated.

The term “comprising” preferably means “consisting of”. In this respect is it particularly preferred that the reaction mix only consists of water, buffer, NAD⁺, PARP, HPF1, sonicated DNA, and the substrate proteins or peptides, all being present in an aqueous buffered solution in the above-indicated concentrations.

The terms “protein” (wherein “protein” is interchangeably used with “polypeptide”) and “peptide” and as used herein describe a group of molecules consisting of amino acids. Whereas peptides consist of up to 30 amino acids, “proteins” consist of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” also refer to naturally modified peptides/proteins wherein the modification consists of e.g. glycosylation, acetylation, phosphorylation and the like. Such modifications are well-known in the art. Peptides and proteins are preferably composed of the 20 naturally occurring amino acids being encoded by the genetic code optionally plus selenocysteine. However, the peptides and proteins may also comprise one or more non-natural amino acids, noting that about 500 amino acids are known in the art. Any non-natural amino acid is preferably an α-amino acids (generic formula H₂NCHRCOOH, where R is an organic substituent known as a “side chain” of the amino acid).

Adenosine diphosphate (ADP; also known as adenosine pyrophosphate (APP)) is an important organic compound in cellular metabolism and is inter alia essential for the flow of energy in living cells. ADP consists of three structural components: a sugar backbone attached to a molecule of adenine and two phosphate groups bonded to the 5′-carbon atom of ribose. ADP-ribose is a related molecular species in which a second ribose is bound via its 5′-carbon to the second phosphate of ADP.

ADP-ribosylation (ADPr) is the addition of one or more ADP-ribose moieties to one or more amino acids of a protein or peptide. In accordance with the present invention the one or more amino acids to which one or more ADP-ribose moieties are added is/are serine(s). Hence, ADP-ribosylation is in accordance with invention serine-ADP-ribosylation (Ser-ADPr). ADPr may be mono-ADP-ribosylation or poly-ADP-ribosylation. Serine-mono-ADP ribosylation is the addition of only one ADP-ribose to a serine side chain. Serine-poly-ADP ribosylation is the addition of two or more ADP-riboses to a serine side chain. The method of the present invention may employ wild-type poly-(ADP-ribose) polymerases (PARPs) that catalyse the transfer of a single or multiple ADP-ribose molecules to target proteins. Hence, the use of wild-type PARPs results in a combination of Ser-mono-ADPr and Ser-poly-ADPr. Means for obtaining exclusively Ser-mono-ADPr proteins and peptides will be discussed herein below. An aqueous solution is a solution in which one of the solvents is water and preferably wherein at least 50% (v/v), more preferably at least 80% (v/v) of the solvents is water. A buffered aqueous solution is an aqueous solution comprising a mixture of a weak acid and its conjugate base, or vice versa. Its pH changes very little when a small amount of strong acid or base is added. Buffered aqueous solutions keep their pH within a certain pH range in a wide variety of chemical applications. This pH range is in accordance with the present invention between 5.0 and 9.0, preferably between 5.5 to 8.5, and most preferably between 6.1 to 8.3. Buffered solutions are necessary to keep the pH within the range, wherein the enzymes used in the method of invention effectively work. If the pH is too high or too low the enzymes may slow or stop working and can even denature.

A buffer (or buffering agent) is accordingly a weak acid or base used to maintain the pH within the above-indicated range after the addition of another acid or base. That is, the function of a buffer is to prevent a rapid change in pH when acids or bases are added to the aqueous buffered solution. A wide range of buffers is available in the art. Buffers can comprise one or more substances. For instance, citric acid can be used as a buffer and the buffer range of citric acid can be extended by adding other buffering agents. McIlvaine buffer is an exemplary buffer composed of citric acid and disodium hydrogen phosphate.

For producing serine ADPr proteins or peptides NAD⁺, one of PARP-1, PARP-2 and PARP-1 E988Q, HPF1, sonicated DNA, and the substrate proteins or peptides to be serine ADP-ribosylated are required.

The amino acid sequence of human PARP-1 is shown in SEQ ID NO: 1, of human PARP-1 E988Q in SEQ ID NO: 2, of mouse PARP-1, isoforms 1 and 2 in SEQ ID NOs 3 and 4 and of rat PARP-1 in SEQ ID NO: 5. The sequence identity of human PARP-1 with mouse and rat PARP-1 is 92%. Accordingly, PARP-1 is preferably a sequence being at least 90% identical to SEQ ID NO: 1. PARP-1 is more preferably a sequence being with increasing preference at least 95%, at least 98% or at least 99% identical to any one of SEQ ID NOs 1 to 5.

The amino acid sequences of human PARP-2, isoforms 1 and 2 are shown in SEQ ID NOs 6 and 7, and of mouse PARP-2 in SEQ ID NO: 8. The sequence identity of human PARP-2 with mouse PARP-2 is 84%. Accordingly, PARP-2 is preferably a sequence being at least 80%, preferably at least 84% identical to SEQ ID NO: 6 or 7. PARP-2 is more preferably a sequence being with increasing preference at least 95%, at least 98% or at least 99% identical to any one of SEQ ID NOs 6 to 8.

The amino acid sequence of human HPF1 is shown in SEQ ID NO: 9 and of mouse HPF1 in SEQ ID NO: 10. The sequence identity of human HPF1 with mouse HPF1 is 89%. Accordingly, HPF1 is preferably a sequence being at least 85%, preferably at least 89% identical to SEQ ID NO: 9. HPF1 is more preferably a sequence being with increasing preference at least 95%, at least 98% or at least 99% identical to SEQ ID NO: 9 or 10.

In accordance with the present invention, the term “percent (%) sequence identity” describes the number of matches (“hits”) of identical amino acids of two or more aligned amino acid sequences as compared to the number of amino acid residues making up the overall length of the template amino acid sequences. In other terms, using an alignment, for two or more sequences or subsequences the percentage of amino acid residues that are the same (e.g. 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. The sequences which are compared to determine sequence identity may thus differ by substitution(s), addition(s) or deletion(s) of amino acids.

The skilled person is also aware of suitable programs to align amino acid sequences. The percentage sequence identity of amino acid sequences can, for example, be determined with programmes such as CLUSTLAW, FASTA and BLAST. Preferably the BLAST programme is used, namely the NCBI BLAST algorithm (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

Nicotinamide adenine dinucleotide (NAD) is a coenzyme found in all living cells. The compound is a dinucleotide consisting of an adenine base and nicotinamide. NAD exists in two forms, an oxidized and reduced form abbreviated as NAD⁺ and NADH respectively. The NAD⁺ used in the method of the invention can be radioactive NAD⁺. The use of radioactive NAD⁺ results in the radioactive labelling of serine ADP-ribosylated peptides and proteins. Herein, the concentration of 0.2 to 2.5 mM NAD⁺ is preferably a concentration of 1.5 to 2.5 mM NAD⁺ and more preferably a concentration of 1.8 to 2.2 mM NAD⁺. Also described herein is a concentration of 1 to 2 mM NAD⁺.

Poly (ADP-ribose) polymerase (PARP) is a family of proteins involved in a number of cellular processes, such as DNA repair, genomic stability, and programmed cell death. Currently the PARP family comprises 17 members (10 putative). The method of the invention employs PARP-1, PARP-2 or the PARP-1 variant E988Q. PARP-1 (also known as NAD⁺ ADP-ribosyltransferase 1 or poly[ADP-ribose] synthase 1) is an enzyme that in humans is encoded by the PARP-1 gene and PARP-2, an enzyme that in humans is encoded by the PARP-2 gene. PARP-1 and PARP-2 contain a catalytic domain and are capable of catalyzing a poly(ADP-ribosyl)ation reaction. PARP-2 has a catalytic domain being homologous to that of PARP-1, but PARP-2 lacks the N-terminal DNA binding domain of PARP-1 which activates the C-terminal catalytic domain of PARP-1. For the action of PARP-1 and PARP-2, NAD⁺ is required as substrate for generating ADP-ribose monomers, as is sonicated DNA, said DNA mimicking DNA with breaks. The PARP-1 E988Q mutant is incapable of poly(ADP-ribosyl)ation activity but instead mono(ADP-ribosyl)ates (Sharifti et al., EMBO J. 2013 May 2; 32(9): 1225-1237). Since the PARP enzyme is the most sensitive compound of the method it is preferably added last to the aqueous solution.

Histone PARylation factor 1 (HPF1) acts as a cofactor for serine ADP-ribosylation by conferring serine specificity on PARP-1 and PARP-2. In more detail, serine ADPr is strictly dependent on histone PARylation factor 1 (HPF1). Quantitative proteomics revealed that histone serine ADPr does not occur in cells lacking HPF1. Moreover, adding HPF1 to in vitro PARP-1/PARP-2 reactions is necessary and sufficient for serine-specific ADPr of histones and PARP-1 itself (Bonfiglio et al., 2017, Mol Cell 65, 932-940 e936).

PARP-1 and -2 are activated by DNA breaks and cleave NAD⁺ thereby generating nicotinamide and ADP-ribose. In higher eukaryotes, PARP-1 and -2 translate the occurrence of DNA breaks detected by its zinc-finger domain into a signal, poly ADP-ribose, synthesized and amplified by its DNA-damage dependent catalytic domain. Hence, the ADPr activity of PARP-1 and -2 requires the presence of DNA breaks. DNA breaks are present in sonicated DNA. Sonicated DNA is DNA that has been fragmented by sonication. Sonication is the act of applying brief periods of ultrasound energy to DNA, so that the DNA is sheared into smaller fragments, preferably fragments of 10 to 330 bp.

The up to 600 μM protein or peptide, said protein or peptide comprising at least one serine, is preferably between 60 and 600 μM protein or peptide, said protein or peptide comprising at least one serine, and is more preferably between 100 and 600 μM protein or peptide, said protein or peptide comprising at least one serine.

In accordance with the present invention the amino acid sequence of the protein or peptide to be modified comprises at least one serine so that by the method of the present invention an ADP-ribosylated protein or peptide is obtained. The at least one serine can be at the C-terminus and/or the N-terminus of the protein or peptide. If present at the N-terminus the serine can be acetylated. Preferably the one or more serines are surrounded by other amino acids.

As discussed herein above, a first small scale in vitro Ser-ADPr reaction on a protein or peptide was performed by the inventors in Bonfiglio et al., 2017, Mol Cell 65, 932-940 e936. However, when subsequently checking the efficiency of the reaction on an inverted poylacylamide gel as described herein below, it was found that by employing the amounts of reagents reported in this publication only a fraction of the set amount of the peptide becomes serine ADP-ribosylated whereas most remains unmodified; see Example 1 and FIG. 1. This also illustrates that the published in vitro Ser-ADPr reaction is not broadly applicable to, for example, other peptide sequences, significantly higher peptide quantities or peptides carrying post-translational modifications. Various variables have to be adjusted in order to optimize the efficiency of the modification, such as the amounts of substrate, PARP-1, HPF1, PARG (if present), NAD+ and the incubation time. Minimization of the amounts of PARP-1, HPF1 and, if present, PARG is especially important considering that these three recombinant proteins are expensive components of the reaction. Therefore, the inventors of the present application had to perform a complex and extensive research program in order to arrive at optimized conditions for serine ADP-ribosylation. Through this process, the inventors arrived at a scalable, efficient reaction that is both high-yielding (maximum amounts of generated serine ADP-ribosylated peptides/proteins with minimum amounts of expensive reagents) and produces highly pure modified peptides (almost 100% of the input substrate peptide gets serine ADP-ribosylated with this reaction) in amounts up to several milligrams; see Examples 1, 2 and 3, and FIG. 1. For potentially poor ADP-ribosylation substrates the efficiency of the reaction can be further boosted by the attachment of a positively-charged tail, such as poly-arginine (FIG. 10). Almost or about 100% purity can be achieved by carrying out the method for the production of a serine ADP-ribosylated protein or peptide under the conditions set forth in the method of the first aspect of the invention and also by using the kit of the second aspect of the invention. Hence, an about 100% efficiency of obtaining serine ADP-ribosylated protein or peptide is achieved.

It should not go unnoticed that in order to produce pure serine ADP-ribosylated peptides, the inventors developed a visualization methodology that allows for the assessment of the efficiency/purity of the modified peptide. Thus it should be emphasized that the development of a simple and effective method of checking the yield, efficiency/purity and the specificity of the reaction was a prerequisite for optimization of the Ser-ADPr-reaction. According to the best knowledge of the inventors, it is not possible by any prior gel electrophoresis methodology to effectively visualize both the ADP-ribosylated peptides/proteins and their unmodified counterparts. The inventors surprisingly found that this is possible by the above-mentioned inverted poylacylamide gel methodology. The inverted poylacylamide gel methodology will be further detailed herein below and is illustrated in Example 4.

In summary, the present invention provides the first means and method for efficient and scalable production of serine ADP-ribosylated proteins and peptides. A scalable high-yield system for generating high amounts of a pure serine ADP-ribosylated version of a given protein or peptide represents a significant advance in the field, since such pure ADP-ribosylated species are required, for example, for the generation of antibodies, the identification of interactors and/or inhibitors, as peptide standards for quantification and optimisation of mass spectrometric approaches, determination of structures, and enzymatic activity profiling. Importantly, also means or methods for the chemical synthesis of serine ADP-ribosylated proteins or peptides cannot be found in the prior art.

In this respect the term “efficiency” or “purity” of the ADP-ribosylation refers to the fraction of the proteins or peptides, said proteins or peptides containing at least one serine, that are serine ADP-ribosylated after the reaction of the method of the invention. The term “about 100% efficiency/purity” means with increasing preference at least 95%, at least 96%, at least 97%, at least 98%, at least 99%, and at least 99.5%. The efficiency/purity is preferably checked by running an inverted polyacrylamide gel as described herein below. An about 100% efficiency/purity is achieved if no band or essentially no band for the unmodified protein or peptide band can be seen by visual inspection of the stained gel, while a band for the serine ADP-ribosylated modified protein or peptide is clearly visible.

In accordance with a preferred embodiment, wherein the solution (constituting the reaction mix) further comprises at least 1 μM, preferably 1 to 10 μM PARG and/or the ADP-ribosylated protein or peptide is incubated with at least 1 μM, preferably 1 to 10 μM PARG, thereby obtaining a serine mono-ADP-ribosylated protein or peptide.

Poly(ADP-ribose) glycohydrolase (PARG) is an enzyme which generates free ADP-ribose from the poly-ADP-ribose chain but cannot cleave the ADP-ribose-protein bond, therefore leading to a serine mono-ADP-ribosylated protein or peptide. Hence, the simultaneous addition of PARG to the reaction mix provides a means for reducing Ser-poly-ADPr into single Ser-mono-ADPr. The amino acid sequences of human PARG, isoforms 1, 2 and 3 are shown in SEQ ID NOs 11, 12 and 13, of mouse PARG, isoforms 1 and 2 in SEQ ID NOs 14 and 15 and of rat PARG in SEQ ID NO: 16. The sequence identity of human PARG with mouse and rat PARG is 87% and 85%, respectively. Accordingly, PARG is preferably a sequence being at least 85% identical to one or more of SEQ ID NOs 11, 12 and 13. PARG is more preferably a sequence being with increasing preference at least 95%, at least 98% or at least 99% identical to any one of SEQ ID NOs 11 to 16.

As is illustrated herein below in Example 3, poly-ADPr of a protein or peptide can be reduced to mono-ADPr via PARG treatment after the protein or peptide has been poly-ADP-ribosylated. ADP-ribosylation may be stopped by the PARP inhibitor Olaparib and poly-ADPr can be reduced to mono-ADPr by directly adding PARG into the solution after the reaction. As an alternative PARG may be added into the solution before the start of the reaction, said solution constituting the reaction mix at the modification step itself. This has the advantage of significantly reducing the overall reaction time. This is because any poly-ADPr is immediately reduced to mono-ADPr in the process of ADP-ribosylation itself; see Example 1 and FIG. 1B. For this reason the option of adding PARG into the solution constituting the reaction mix is preferred.

As detailed herein above, an alternative strategy could be employed by using the PARP-1 mutant E988Q, which only mono-ADP-ribosylates and, therefore, produces Ser-mono-ADPr under the experimental conditions described herein. Hence, in case PARG is present it is preferred to use PARP-1 or PARP-2 instead of the PARP-1 mutant E988Q.

In accordance with a preferred embodiment, the reaction is carried out at 15-35° C. and preferably at room temperature.

As part of the optimization of the conditions of the Ser-ADP-ribosylation of proteins or peptides the reaction temperature was also tested. The reaction is very robust and works at temperatures that might be found in a laboratory, i.e. 15-35° C. Room temperature preferably refers to a temperature range of 18-27° C. and more preferably 20-25° C.

In accordance with a further preferred embodiment, the reaction is carried out for at least 90 min, preferably at least 120 min, more preferably at least 240 min, and most preferably at least 320 min.

The reaction time was also a factor in the optimization of the conditions of the Ser-ADP-ribosylation of proteins or peptides. It was found that it is preferred to carry out the reaction for at least 90 min in order to effectively ADP-ribosylate the target serine/s of the protein or peptide. In particular for longer proteins/peptides carrying many serines it may be advantageous to prolong the reaction time to at least 120 min or at least 240 min or at least 320 min.

In accordance with a further preferred embodiment, a fresh pool of 0.2 to 2.5 mM NAD⁺ is added to the reaction mix at least every 90 minutes, preferably at least every 60 minutes and more preferably at least every 45 minutes.

Also here the concentration of 0.2 to 2.5 mM NAD⁺ is preferably a concentration of 1.5 to 2.5 mM NAD⁺ and more preferably a concentration of 1.8 to 2.2 mM NAD⁺. Also described herein is a concentration of 1 to 2 mM NAD⁺. Connected with the reaction time, the addition of a fresh pool of NAD⁺ was tested during the optimization of the conditions of the Ser-ADP-ribosylation of proteins or peptides. It was found that it is preferred to add fresh NAD⁺ to the solution constituting the reaction mix at least every 90 minutes in order to effectively ADP-ribosylate the target serine/s of the protein or peptide. In particular for specific substrates, it may be advantageous to add fresh NAD⁺ to the reaction mix at least every 60 minutes or 45 minutes.

After the reaction is carried out it could be stopped by the addition of a PARP inhibitor. The PARP inhibitor is preferably Olaparib, and Olaparib is preferably used at a concentration of about 2 μm. In this connection the term “about” is preferably ±20% and more preferably ±10%. Another example of a PARP inhibitor is Rucaparib.

In accordance with another further preferred embodiment, the at least one serine is neighboured by at least one basic amino acid.

The at least one basic amino acid is with increasing preference at least two, at least three and at least four basic amino acids. The basic amino acids are preferably Lys and/or Arg. The term “neighboured” means that at least one basic amino acid can be found up to three amino acids adjacent to the serine to be ADPr, either N-terminally or C-terminally (i.e. at amino acid positions −3 to +3). Non-limiting examples of such a motif are “KAASAAA” and “AAASARA”. The at least one basic amino acid is preferably up to two amino acids adjacent to the serine to be ADPr (i.e. at amino acid positions −2 to +2) and more preferably directly adjacent to the serine (i.e. at amino acid positions −1 and/or +1). The motif “KS” (i.e. Lysine at the −1 amino acid position) is most preferred since it can be frequently found in nature. Among all these options it is preferred that the serine is neighboured on both sides by at least one basic amino acid.

Related to the above further preferred embodiment it is preferred that the protein or peptide to be ADP-ribosylated has an isoelectric point of at least 8.0.

The isoelectric point is the pH at which a particular molecule carries no net electrical charge in the statistical mean. Among the naturally occurring amino acids the basic amino acids Lys and Arg have the highest isoelectric point. The isoelectric point of Lys is 9.74 and the isoelectric point of Arg is 10.76. Hence, a protein or peptide having an isoelectric point of at least 8.0 is a protein or peptide that carries no net electrical charge at a basic pH of 8.0 or higher.

It has been found that within the proteins or peptides the serines being neighboured by at least one basic amino acid as well as peptides having an overall basic isoelectric point of at least 8.0 are particularly well recognized by the Ser-ADP-ribosylation machinery. The same holds true for the following preferred embodiment.

In accordance with a yet further preferred embodiment, the substrate contains a positively charged tail, preferably a poly-arginine and/or lysine tail.

The term “positively charged tail” refers to a C- or N-terminal tail of the peptide or protein to be Ser-ADPr, which comprises or consists of at least 3 basic amino acids, preferably Lys and/or Arg residues. The positively charged tail preferably comprises or consists of at least 4 basic amino acids, preferably Lys and/or Arg residues. Also the poly-Lys or poly-Arg tail can be attached to the N-terminus or the C-terminus of the substrate peptide or protein to be ADPr. It was surprisingly found that for specific substrates, the addition of a positively charged tail further boosts the reaction efficiently; see FIG. 10.

In accordance with a yet further preferred embodiment, the aqueous buffered solution further comprises (e) 10 to 80 mM NaCl or KCl, and/or (l 0.5 to 2 mM MgCl₂.

The activity of enzymes may be affected by the addition of inorganic salts, e.g., during in vitro assays. The concentration of salts, the identity of the ions, and the ionic strength of the solution can affect the activity of an enzyme. In connection with the optimization of the conditions of the Ser-ADP-ribosylation of proteins or peptides it was surprisingly found that the enzymatic activity of PARP does not require the presence of inorganic salts, so that peptides and protein can be highly efficiently Ser-ADP-ribosylated in the absence of salts in the aqueous solution. However, it is also possible to use PARP in connection with inorganic salts. A non-limiting example of inorganic salt conditions is the use of 10 to 80 mM NaCl or KCl, and/or 0.5 to 2 mM MgCl₂ in the aqueous solution.

In accordance with a preferred embodiment, the buffer is a Tris-HCl buffer and is preferably used at a final concentration of 40-60 mM, a Hepes buffer and is preferably used at a final concentration of 40-60 mM, more preferably about 50 mM, or a phosphate buffer and is preferably used at a final concentration of 5-20 mM, more preferably about 10 mM.

Tris has a pKa of 8.07 at 25° C., which implies that Tris-HCl buffer has an effective pH range between 7.5 and 9.0. The useful buffer range for Tris-HCl (7.5 to 9) coincides with the physiological pH typical of most living organisms. This and its low cost make Tris-HCl one of the most common buffers in the biology/biochemistry laboratory. In this respect it is noted that the pH of a solution is defined as the negative logarithm to the base 10 of the hydrogen ion concentration in g ions L⁻¹ (pH=−log 10 [H⁺]). Thus pH can also be defined as the logarithm to the base 10 of the reciprocal of H⁺ ion concentration. During the ionization of weak acid HA, where the ionization is not complete, the dissociation constant is the ratio of the dissociated and undissociated components (Ka=[H⁺]×[A⁻]/[HA]). Hence, pKa is defined as the negative logarithm to the base 10 of the Ka in g ions L⁻¹ or as logarithm to the base 10 of the reciprocal of Ka. The relationship between pH and pKa is given by Henderson-Hasselbach equation (pH=pKa+log[A⁻]/[HA]).

HEPES (4-(2-hydroxyethyl)-1-piperazineethanesulfonic acid) is a zwitterionic organic chemical buffering agent. HEPES has a pKₐ₁ (25 C)=3 and a pKₐ₂ (25 C)=7.5 and accordingly has buffering capacity at pH ranges between 2.5 to 3.5 and 6.8 to 8.2. The latter pH range coincides with the physiological pH typical of most living organisms.

Phosphates have a very high buffering capacity and are highly soluble in water. Gomori buffers, the most commonly used phosphate buffers, consist of a mixture of monobasic dihydrogen phosphate and dibasic monohydrogen phosphate. By varying the amount of each salt, a range of buffers can be prepared that buffer well between pH 5.8 and 8.0.

In accordance with another preferred embodiment, the aqueous solution is water.

As mentioned above, an aqueous solution is a solution in which one of the solvents is water. In this respect it is preferred that water is the only solvent so that the aqueous solution is water.

In accordance with a further preferred embodiment, the method further comprises purifying the serine ADP-ribosylated protein or peptide from the reaction mix.

By the end of the method of the present invention, the reaction mix comprises the serine ADPr proteins or peptides and in addition at least NAD⁺, one of PARP-1, PARP-2 and PARP-1 E988Q, HPF1, sonicated DNA and, if present, PARG. In accordance with the above preferred embodiment the Ser-ADPr proteins or peptides are purified from these additional compounds. Means and methods for purifying the serine ADP-ribosylated protein or peptide from the reaction mix are known in the art and preferred examples will be further detailed herein below.

In accordance with a more preferred embodiment, the serine ADP-ribosylated protein or peptide is purified from the reaction mix by StageTip fractionation employing C8, C18, SCX, SAX or SDB-RPS chromatography media, cation or anion exchange chromatography, hydrophilic interaction chromatography (HILIC), phosphopeptide enrichment, enrichment with a ADP-ribose-binding protein domain, boronate affinity chromatography, filtering the reaction with an ultrafiltration device, a spin column or a combination thereof.

Stage tips can be used to rapidly purify proteins and peptides from a reaction mix. C8, C18, SAX and SCX stage tips are commercially available. C8, C13, SAX, SCX and SDB-RPS are chromatography materials and these materials are loaded into pipet tips. C8 and 018 are silica-based materials. SCX and SAX refer to strong cation exchange and strong anion exchange resins and membranes, respectively. SDB-RPS is a styrenedivinylbenzene resin that has been modified with sulfonic acid groups to make it hydrophilic. Because SDB-RPS displays both reversed phase and cation exchange interactions, both affinities can be considered to design selective extractions.

Cation and anion exchange chromatography are forms of ion exchange chromatography, which can be used to separate molecules based on their net surface charge. Cation exchange chromatography uses a negatively charged ion exchange resin with an affinity for molecules having net positive surface charges. Anion-exchange chromatography is a process that separates substances based on their charges using an ion-exchange resin containing positively charged groups, such as diethyl-aminoethyl groups (DEAE). Cation and anion exchange chromatography are used both for preparative and analytical purposes and can separate a large range of molecules, including serine ADP-ribosylated proteins or peptides.

Hydrophilic interaction chromatography (or hydrophilic interaction liquid chromatography, HILIC) is a variant of normal-phase liquid chromatography that partly overlaps with other chromatographic applications such as ion chromatography and reversed phase liquid chromatography. HILIC uses hydrophilic stationary phases with reversed-phase type eluents.

Phosphopeptide enrichment enables efficient enrichment of phosphorylated peptides and proteins from complex and fractionated protein digests. Since the ADP-ribosylated protein or peptide contains phosphate groups, phosphopeptide enrichment can be used for its purification. For example, TiO₂ and Fe-NTA phosphopeptide enrichment kits are commercially available.

ADP-ribose-binding domains are domains of proteins that bind to ADP-ribose moieties (Vivelo and Leung (2015), Proteomics. 2015 January; 15(0): 203-217). Thus these domains can be used to purify ADP-ribosylated proteins or peptides. Several protein domains that bind mono- and poly(ADP-ribose) are known, such as the WWE domain, the PBZ (PAR-binding zinc finger) domain, the PBM (PAR-binding motif), the Af1521 macrodomain, and the catalytically inactive E756D mutant of PARG (PARG-DEAD approach).

Boronate affinity chromatography is a unique means for selective separation and enrichment of cis-diol-containing compounds. Cis-diol-containing biomolecules are an important class of compounds, including glycoproteins, glycopeptides, ribonucleosides, ribonucleotides, saccharides, and catecholamines.

Ultrafiltration devices are commercially available for the concentration of biological samples. They can be used, for example, in either a swing bucket or fixed angle rotors which accept 2.0 mL centrifuge tube at maximum speed 10,000×g. During centrifugation the serine ADP-ribosylated peptides flow through the filter whereas other ingredients of the reaction mix, such as PARP and HPF are retained on the filter.

Spin columns, such as the G25 spin columns are microspin columns that were initially designed for the rapid purification of DNA for use in a wide range of applications, including desalting, buffer exchange, and removal of unincorporated nucleotides from end-labelled oligonucleotides. When used in connection with serine ADP-ribosylated proteins or peptides as comprised in the reaction mix other ingredients of the reaction mix, such as NAD⁺, can be removed.

In accordance with a more preferred embodiment, the efficiency of the serine ADP-ribosylation of the protein or peptide is checked by running the reaction product on a polyacrylamide gel with inverted polarity.

As described herein above, the method of the invention results in essentially 100% efficiency of obtaining Ser-ADP-ribosylated proteins and peptides. The above preferred embodiment is to control whether this completeness of serine ADP-ribosylation has indeed been achieved for a particular batch of Ser-ADP-ribosylated proteins and peptides. This is important to ensure production quality control, since in the laboratory practice one or more of the reagents of the reaction might not work properly. For instance, the enzyme PARP may have a reduced activity if is too old or was stored at too high temperature.

To the best knowledge of the inventors the possibility of checking the efficiency, yield and specificity of the serine ADP-ribosylation of the protein or peptide by running a serine ADP-ribosylated protein or peptide on a polyacrylamide gel with inverted polarity is not described in the state of the art.

For this reason the present invention also relates to a method for checking the efficiency, and/or yield and/or specificity of the serine ADP-ribosylation of the protein or peptide by running a serine ADP-ribosylated protein or peptide, preferably obtained with the method of the invention on a polyacrylamide gel with inverted polarity.

The inventors found that unmodified and Ser-ADPr modified peptides can be separated from each other by running them on a polyacrylamide gel with inverted polarity. On the polyacrylamide gel with inverted polarity the positively charged peptides run into the gel and are separated according to their charge. The basic principle of this separation is that ADP-ribosylated peptides are less positively charged compared to their unmodified counterparts and therefore migrate more slowly. After the gel run, any peptide/protein staining strategy can be used, and both separated species—the unmodified and the Ser-ADPr-modified peptides or proteins—can be detected. Staining strategies are known in the art and preferred staining strategies will be described herein below.

In connection with all above-described methods for checking the efficiency of the serine ADP-ribosylation the polyacrylamide gel is preferably a Tris/Borate/EDTA (TBE) polyacrylamide gel. Moreover, into the methods a negative control may be implemented to check the site specificity of the reaction, preferably on the same gel in a different gel lane. The negative control can be a protein or peptide with a sequence resembling that of the protein or peptide to be serine ADP-ribosylated, in which the serines have been replaced by any other amino acid, preferably alanines. In addition, the serine specificity of the ADPr reaction can be checked by employing a negative control which can be a portion of the serine ADP-ribosylated protein or peptide that has been treated with an enzyme that specifically removes serine ADP-ribosylation from the peptide or protein. Such enzyme is preferably a ADP-ribose hydrolase being capable of removing serine ADP-ribosylation from the peptide or protein and is most preferably ADP-ribose hydrolase 3 (ARH3) (Fontana et al., eLife, 2017; 6:e28533). The amino acid sequence of human ARH3 is shown in SEQ ID NO: 17 and of mouse ARH3, isoforms 1 and 2 in SEQ ID NOs 18 and 19. The sequence identity of human ARH3 with mouse ARH3 is 92%. Accordingly, ARH3 is preferably a sequence being at least 90% identical to SEQ ID NO: 18 or 19. ARH3 is more preferably a sequence being with increasing preference at least 95%, at least 98% or at least 99% identical to any one of SEQ ID NOs 17 to 19. Moreover, the negative control may also be a protein or peptide which corresponds to the protein or peptide to be serine ADP-ribosylated, wherein the serines were not modified by ADP-ribosylation.

In accordance with a more preferred embodiment, the protein or peptide is stained, preferably with a Coomassie dye, silver staining or a reverse staining technique, such as imidazole reverse staining and zinc reverse staining, and is more preferably stained with Imperial™ protein stain.

Coomassie dye (or Coomassie Brilliant Blue) refers to two similar triphenylmethane dyes that have been used for staining proteins in analytical biochemistry since the 1960s. Coomassie Brilliant Blue G-250 differs from Coomassie Brilliant Blue R-250 by the addition of two methyl groups. Imperial™ protein stain has been used in the examples herein below. It is a coomassie R-250 dye-based reagent for protein staining in polyacrylamide gels. This sensitive (3 ng) stain produces an intense color that photographs well. This reagent stains only protein and allows bands to be viewed directly in the gel during the staining process.

Silver staining is used to detect proteins after electrophoretic separation on polyacrylamide gels. It combines excellent sensitivity (in the low nanogram range) whilst using very simple and cheap equipment and chemicals (Chevallet et al., Nat Protoc. 2006; 1(4): 1852-1858).

Also imidazole reverse staining and zinc reverse staining are widely used for the staining of peptides or proteins in polyacrylamide gels. Imidazole/zinc reverse stain is known for its high sensitivity, ease of use, and cost-effective feature (Chen, Methods Mol Biol. 2012; 869:487-95).

In accordance with a more preferred embodiment, the method is carried out in vitro or ex vivo.

In vitro methods (i.e. in the glass) are performed with microorganisms, isolated cells, or biological molecules outside their normal biological context. For example, microorganisms or cells can be studied in artificial culture media, and proteins can be examined in solutions. Ex vivo methods are carried out outside a living-animal.

In accordance with a further preferred embodiment, the method may additionally comprise the step of formulating the produced serine ADP-ribosylated protein or peptide into a pharmaceutical composition.

In accordance with the present invention, the term “pharmaceutical composition” relates to a composition for administration to a patient, preferably a human patient. As mentioned, the pharmaceutical composition of the invention comprises the produced serine ADP-ribosylated proteins or peptides. It may, optionally, comprise further molecules capable of altering the characteristics of said proteins or peptides thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician.

The present invention relates in a second aspect to a kit for the production of a serine ADP-ribosylated protein or peptide comprising (a) 5 to 60 mM, preferably 10 to 60 mM of a buffer having a pKa between 5.0 and 9.0, preferably between 5.5 and 8.5, and most preferably between 6.1 to 8.3, (b) 0.2 to 2.5 mM NAD⁺, (c) at least 50 nM, preferably 50 to 3000 nM PARP-1, PARP-2 or the PARP-1 variant, (d) at least 100 nM, preferably 100 to 5000 nM HPF1, (e) at least 10 μg/mL, preferably 10 μg/mL to 200 μg/mL sonicated DNA, said sonicated DNA preferably comprising DNA fragments of 10 to 330 bp, (f) optionally at least 1 μM, preferably 1 to 10 μM PARG, (g) optionally 10 to 80 mM NaCl or KCl, and (h) optionally 0.5 to 2 mM MgCl₂, in one or more container(s).

The kit of the invention comprises the components required to carry out the method of the first aspect of the invention packed into one or more container(s), with exception of the protein or peptide to be ADP-ribosylated. For this reason the definitions and the preferred examples and concentrations of the components as describes herein above in connection with the first aspect of the invention apply mutatis mutandis to the second aspect of the invention. For example, also in connection with the kit the concentration of 0.2 to 2.5 mM NAD⁺ is preferably a concentration of 1.5 to 2.5 mM NAD⁺ and more preferably a concentration of 1.8 to 2.2 mM NAD⁺.

The one or more containers may be, for example, one or more vials. The vials may, in addition to the components, comprise preservatives or buffers for storage. In addition, the kit may contain instructions for use.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

FIG. 1—(A) Comparison of the efficiency of the reaction under different conditions (Lane 1) 500 μM Untreated H3 (1-22) peptide, (Lane 2) 500 μM H3 (1-22) peptide reacted under the conditions used in P32-NAD experiments in Bonfiglio et al., 2017, Mol Cell 65, 932-940 e936 (5.5 μM NAD⁺, 0.1 μM PARP-1, 2 μM HPF1 for 20′ RT), (Lane 3) 500 μM H3 (1-22) peptide reacted under the conditions used in MSMS experiments Bonfiglio et al., 2017, Mol Cell 65, 932-940 e936 (200 μM NAD⁺, 0.1 μM PARP-1, 2 μM HPF1 for 20′ RT), (Lane 4) 500 μM H3 (1-22) peptide reacted under optimized conditions used to illustrate the present invention (2 mM NAD⁺, 0.05 μM PARP-1, 500 nM HPF1 for 2 h RT). Note the reduced amount of PARP-1 and HPF1 used in lane 4 compared to lanes 2 and 3. (B) Comparison of the efficiency of the reaction under different conditions (Lane 1) 62 μM Untreated H2B (1-22) peptide, (Lane 2) 62 μM H2B (1-22) peptide reacted under the conditions used in P32-NAD experiments in Bonfiglio et al., 2017, Mol Cell 65, 932-940 e936 (5.5 μM NAD⁺, 0.1 μM PARP-1, 2 μM HPF1 for 20′ RT), (Lane 3) 62 μM H2B (1-22) peptide reacted under the conditions used in MSMS experiments Bonfiglio et al., 2017, Mol Cell 65, 932-940 e936 (200 μM NAD⁺, 0.1 μM PARP-1, 2 μM HPF1 for 20′ RT), (Lane 4) 62 μM H3 (1-22) peptide reacted under optimized conditions used to illustrate the present invention (0.1 μM PARP-1, 500 nM HPF1, 1 μM PARG, adding 200 μM NAD⁺ every 1 hour for up to 6 hours). (C) For specific substrates the addition of a positively charged tail boosts the efficiency of the reaction (Lane 1) 83 μM Untreated H2B (1-22) peptide, (Lane 2) 83 μM H2B (1-22) peptide reacted with 200 μM NAD⁺, 0.1 μM PARP-1, 2 μM HPF1 for 30′ RT, (Lane 3) 66 μM Untreated H2B (1-22) peptide C′terminal poly(Arg) tail, (Lane 4) 66 μM H2B (1-22) peptide C′terminal poly(Arg) tail reacted with 200 μM NAD⁺, 0.1 μM PARP-1, 2 μM HPF1 for 30′ RT. (D) Comparison of the efficiency of the reaction under different conditions (Lane 1) 64 μM Untreated Histone H4 (1-19)-Biotin peptide, (Lane 2) 64 μM Histone H4 (1-19)-Biotin peptide reacted under the conditions used in P32-NAD experiments in Bonfiglio et al., 2017, Mol Cell 65, 932-940 e936 (5.5 μM NAD⁺, 0.1 μM PARP-1, 2 μM HPF1 for 20′ RT), (Lane 3) 64 μM Histone H4 (1-19)-Biotin peptide reacted under the conditions used in MSMS experiments Bonfiglio et al., 2017, Mol Cell 65, 932-940 e936 (200 μM NAD⁺, 0.1 μM PARP-1, 2 μM HPF1 for 20′ RT), (Lane 4) 64 μM Histone H4 (1-19)-Biotin peptide reacted under optimized conditions used to illustrate the present invention (0.1 μM PARP-1, 500 nM HPF1, 1 μM PARG, adding 200 μM NAD⁺ every 1 hour for up to 6 hours).

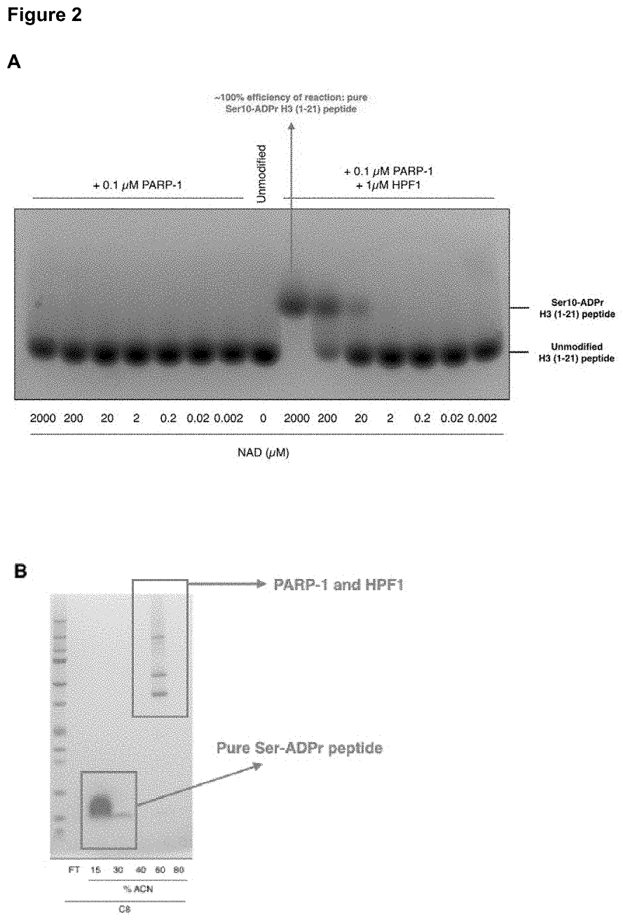

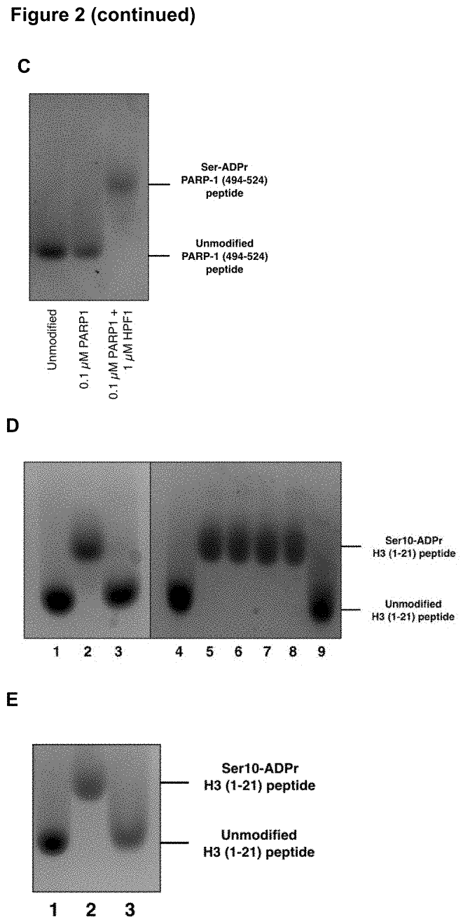

FIG. 2—(A) TBE sequencing gel run with inverted polarity and stained with Imperial™ Protein Stain (Thermo). Contrary to what occurs when using radioactivity with standard SDS-PAGE, both unmodified and Ser-ADPr histone H3 (1-21) peptides can be resolved and visualized by this novel strategy. By this method, the inventors were able to test different conditions to optimize the efficiency of the reaction (NAD⁺, substrate, PARP-1 and HPF1 concentrations, buffer compositions, incubation times). As shown with this particular example, after testing different concentrations of NAD⁺ in the presence or absence of 1 μM HPF1, a condition was found in which after a 90 minutes reaction, ˜100% of the peptide (110 μM) is ADP-ribosylated on serine. (B) SDS-PAGE Tricine gel run with normal polarity and stained by InstantBlue™ Ultrafast Protein Stain (SIGMA). Although this commonly-used methodology does not permit discrimination between Ser-ADPr and unmodified peptides, it is useful to determine the purity of the sample. As depicted in the figure the modified Ser-ADPr peptides can be completely separated from the other components that were present in the in vitro ADPr reaction (PARP-1 and HPF1). Importantly, although not shown, NAD⁺ is also removed by this separation strategy. (C) TBE sequencing gel run with inverted polarity and stained with Imperial™ Protein Stain (Thermo). Comparable to what is shown in panel A, Ser-ADPr PARP-1 (494-524) peptides can be resolved by this novel visualization strategy (TBE sequencing gel run with inverted polarity). With this example, the inventors demonstrate that the optimized conditions used to illustrate the present invention require adding HPF1 to the reaction mix (2 mM NAD⁺, 0.05 μM PARP-1) and incubating for 90 minutes to obtain ˜100% Ser-ADP-ribosylation of 110 μM of a PARP-1 (494-524) peptide. (D) TBE sequencing gel run with inverted polarity and stained with Imperial™ Protein Stain (Thermo). Comparable to what is shown in panel A, inventors performed in vitro ADP-ribosylation assays under different experimental conditions in order to optimize the yield and efficiency of the reaction. (Lane 1) 200 μM Untreated H3 (1-22) peptide, (Lane 2) 200 μM H3 (1-22) peptide reacted with 2 mM NAD⁺, 0.1 μM PARP-1, 2 μM HPF1 in the presence of activated DNA for 2 hours RT, (Lane 3) 200 μM H3 (1-22) peptide reacted with 2 mM NAD⁺, 0.1 μM PARP-1, 2 μM HPF1 in the absence of activated DNA for 2 hours RT, (Lane 4) 82 μM Untreated H3 (1-22) peptide, (Lane 5) 82 μM H3 (1-22) peptide reacted with 2 mM NAD⁺, 0.1 μM PARP-1, 500 nM HPF1 in 50 mM Tris pH: 7.5, 50 mM NaCl, 1 mM MgCl₂, for 2 hours RT, (Lane 6) 82 μM H3 (1-22) peptide reacted with 2 mM NAD⁺, 0.1 μM PARP-1, 500 nM HPF1 in 40 mM Tris pH: 7.5, 50 mM NaCl, 1 mM MgCl₂, for 2 hours RT, (Lane 7) 82 μM H3 (1-22) peptide reacted with 2 mM NAD⁺, 0.1 μM PARP-1, 500 nM HPF1 in 60 mM Tris pH: 7.5, 50 mM NaCl, 1 mM MgCl₂, for 2 hours RT, (Lane 8) 82 μM H3 (1-22) peptide reacted with 2 mM NAD⁺, 0.1 μM PARP-1, 500 nM HPF1 in 50 mM Tris pH: 7.5, 10 mM NaCl, 1 mM MgCl₂, for 2 hours RT, (Lane 9) 82 μM H3 (1-22) peptide reacted with 2 mM NAD⁺, 0.1 μM PARP-1, 500 nM HPF1 in 50 mM Tris pH: 7.5, 100 mM NaCl, 1 mM MgCl₂, for 2 hours RT. (E) The serine specificity of the ADPr reaction can be checked by treating a portion of the serine ADP-ribosylated protein or peptide with an enzyme that specifically removes serine ADP-ribosylation from the peptide or protein. Such enzyme is preferably ARH3. (Lane 1) 74 μM Untreated Histone H3 (1-21) peptide, (Lane 2) 74 μM Histone H3 (1-21) peptide reacted with 2 mM NAD⁺, 0.1 μM PARP-1 and 2 μM HPF1, for 2 hours RT (Lane 3) Half of the reaction from Lane 2 was incubated with 0.5 μM ARH3 for 30 minutes at RT.

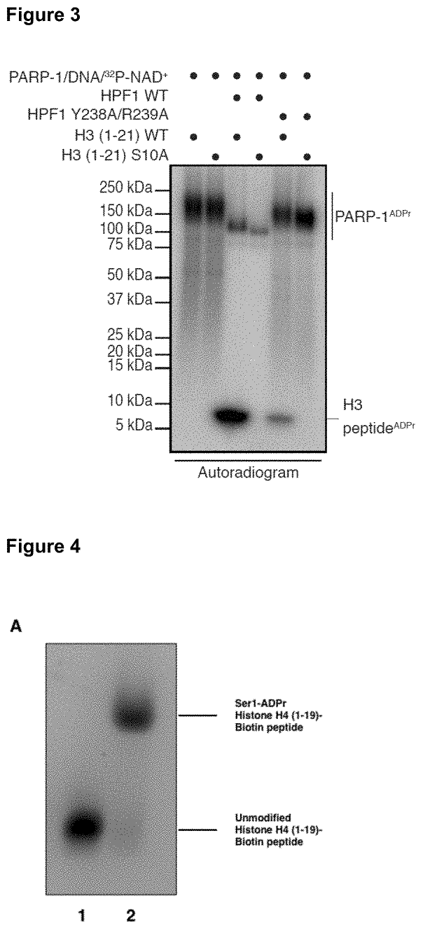

FIG. 3—Autoradiogram showing ADP-ribosylation of two synthetic peptide variants corresponding to amino acids 1-21 of human H3. To generate these modified peptides, we performed an in vitro ADPr reaction using PARP-1, HPF1, and radioactive (32P) NAD⁺. As depicted in the figure, only species with 32P radioactivity (ADPr species) are detected. Unmodified species cannot be detected as they are not radioactively labelled, which prevents any estimation of the efficiency of the reaction and, in the same line, the purity of the species present (FIG. 3 is taken from in Bonfiglio et al., 2017, loc. lit.).

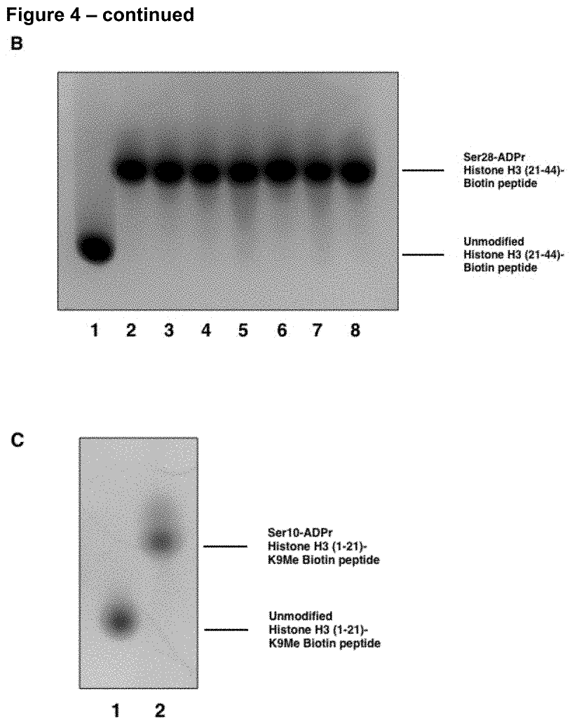

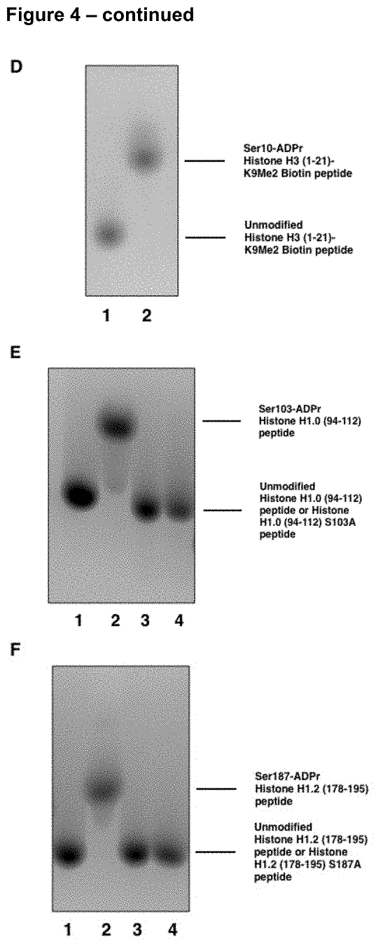

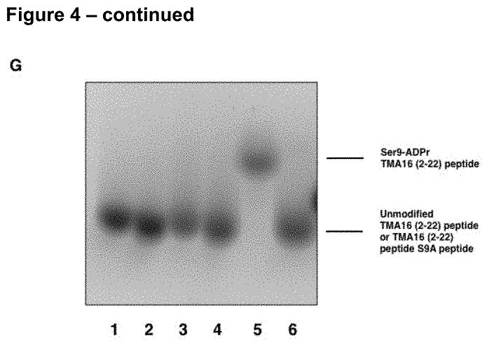

FIG. 4—TBE sequencing gels run with inverted polarity and stained with Imperial™ Protein Stain (Thermo). To further demonstrate that the claimed 100% serine ADP-ribosylation method is applicable to virtually any peptide or protein with an amino acid sequence containing or comprising at least one serine, the inventors have applied the present invention on different substrates. (A) Histone H4 (1-19)-Biotin peptide (Lane 1) 170 μM Untreated Histone H4 (1-19)-Biotin peptide, (Lane 2) 170 μM Histone H4 (1-19)-Biotin peptide reacted with 2 mM NAD⁺, 0.12 μM PARP-1, 1.5 μM HPF1 and 1 μM PARG in the presence of activated DNA for 6 hours RT. (B) Histone H3 (21-44)-Biotin peptide (Lane 1) 195 μM Untreated Histone H3 (21-44)-Biotin peptide, (Lanes 2 to 8) Different replicates for 195 μM Histone H3 (21-44)-Biotin peptide reacted with 2 mM NAD⁺, 0.12 μM PARP-1 and 1 μM HPF1 in the presence of activated DNA for 5 hours RT. (C) Histone H3 (1-21)-K9Me Biotin peptide (Lane 1) 95 μM Untreated Histone H3 (1-21)-K9Me Biotin peptide, (Lane 2) 95 μM Histone H3 (1-21)-K9Me Biotin peptide reacted with 2 mM NAD⁺ every 2 h, 0.1 μM PARP-1 and 1 μM HPF1, in the presence of activated DNA for 6 hours RT. (D) Histone H3 (1-21)-K9Me2 Biotin peptide (Lane 1) 95 μM Untreated Histone H3 (1-21)-K9Me2 Biotin peptide, (Lane 2) 95 μM Histone H3 (1-21)-K9Me2 Biotin peptide reacted with 2 mM NAD⁺ every 2 h, 0.1 μM PARP-1 and 1 μM HPF1, in the presence of activated DNA for 6 hours RT. (E) Histone H1.0 (94-112) peptide (Lane 1) 90 μM Histone H1.0 (94-112) peptide, (Lane 2) 90 μM Histone H1.0 (94-112) peptide reacted with 2 mM NAD⁺, 0.1 μM PARP-1 and 0.5 μM HPF1 in the presence of activated DNA for 2 hours RT, (Lane 3) 90 μM Histone H1.0 (94-112) S103A peptide, (Lane 4) 90 μM Histone H1.0 (94-112) S103A peptide reacted with 2 mM NAD⁺, 0.1 μM PARP-1 and 0.5 μM HPF1 in the presence of activated DNA for 2 hours RT. (F) Histone H1.2 (178-195) peptide (Lane 1) 100 μM Histone H1.2 (178-195) peptide, (Lane 2) 100 μM Histone H1.2 (178-195) peptide reacted with 2 mM NAD⁺, 0.1 μM PARP-1 and 0.5 μM HPF1 in the presence of activated DNA for 2 hours RT, (Lane 3) 100 μM Histone H1.2 (178-195) S187A peptide, (Lane 4) 100 μM Histone H1.2 (178-195) S187A peptide reacted with 2 mM NAD⁺, 0.1 μM PARP-1 and 0.5 μM HPF1 in the presence of activated DNA for 2 hours RT. (G) TMA16 (2-22) peptide (Lane 1) 115 μM Untreated TMA16 (2-22) peptide, (Lane 2) 115 μM Untreated TMA16 (2-22) S9A peptide, (Lane 3) 115 μM TMA16 (2-22) peptide reacted with 2 mM NAD⁺ and 0.1 μM PARP-1 in the presence of activated DNA for 6 hours RT, (Lane 4) 115 μM TMA16 (2-22) S9A peptide reacted with 2 mM NAD⁺ and 0.1 μM PARP-1 in the presence of activated DNA for 6 hours RT, (Lane 5) 115 μM TMA16 (2-22) peptide reacted with 2 mM NAD⁺, 0.1 μM PARP-1 and 1.5 μM HPF1 in the presence of activated DNA for 6 hours RT, (Lane 6) 115 μM TMA16 (2-22) S9A peptide reacted with 2 mM NAD⁺, 0.1 μM PARP-1 and 1.5 μM HPF1 in the presence of activated DNA for 6 hours RT.

The examples illustrate the invention.

### EXAMPLE 1—COMPARISON OF THE EFFICIENCY OF THE REACTION UNDER DIFFERENT CONDITIONS

The present invention provides means and methods for the efficient and scalable production of essentially pure Ser-ADPr proteins or peptides. This scalable system for high-yield and efficient generation of the pure Ser-ADP-ribosylated version of a given protein or peptide represents a significant advance in the field. As revealed in FIGS. 1A, B and D, the in vitro modification of different synthetic peptides performed under the experimental conditions published in the state of the art (Bonfiglio et al., 2017, Mol Cell 65, 932-940 e936) does not result in the complete Ser-ADPr of the peptide, in particular no pure Ser-ADP-ribosylated version of a protein or a peptide can be obtained. In contrast, by reacting the same synthetic peptides under the optimized conditions used to illustrate the present invention, a ˜100% pure Ser-ADPr protein or peptide can be obtained. A complementary advantage of the present invention is that by evaluating the efficiency of the reaction (see Example 4), the inventors were able to determine and set forth the experimental conditions to obtain the highest amount of pure Ser-ADPr peptide using the smallest amount of expensive reagents, in particular PARP-1, HPF1 and PARG, if present.

### EXAMPLE 2—TESTED CONDITIONS UNDER WHICH AN ABOUT 100% EFFICIENCY WAS OBTAINED

Among the tested conditions the following conditions resulted in about 100% efficiency:

### Solvent:

### Water

### Buffer:

(i) 40-60 mM Tris-HCl, pH=7.5; (ii) 50 mM Hepes pH=7.5; or (iii) 10 mM phosphate buffer (pHs=6.1, 6.6, 7.2, 7.7, 8.3);

### Salt:

(i) No salts, or (ii) 10-80 mM NaCl and/or 50 mM KCl (iii) and/or 0.5-2 mM MgCl₂;

### NAD⁺: 1.8 to 2.2 mM;

PARP-1: at least 50 nM PARP-1;
HPF1: at least 100 nM;
Substrate peptide: 100 to 600 μM (e.g. histone H3 (1-21) peptides);
Incubation time: at least 90 min;
PARG: if present, at least 1 μM

The required elements for producing Ser-ADPr peptides are PARP-1, HPF1, NAD⁺, activated DNA and the substrate itself, all of them contained in a reactor (e.g. tube) in which the modification of the substrate occurs. As the in vitro modification of different synthetic peptides performed under the experimental conditions published in the state of the art does not result in the complete Ser-ADPr of the peptide (FIGS. 1A, B and D), different variables needed to be adjusted in order to optimize the efficiency of the modification, such as the incubation time, the amount of substrate, the amount of PARP-1, HPF1, PARG (if present), and/or NAD⁺. Using the visualization methodology described in Example 4, the production of the ADPr version for various substrate peptides was optimized and ˜100% of the modification was reached under the above shown conditions. After reaching ˜100% of the modification (FIGS. 2A, 2C and 2D), a cleaning step can be carried out to eliminate all the reacting elements except the Ser-ADPr peptide. For the cleaning step, for example, a stage tip with a C8 resin that enables fast and efficient purification of the peptide can be used (FIG. 2B). To note, all of the materials required are also available in a much larger scale to make much higher levels of production possible (see Example 3).

### EXAMPLE 3—EXPERIMENTAL CONDITIONS UNDER WHICH LARGE AMOUNTS OF PURE SERINE MONO-ADP-RIBOSYLATED PEPTIDE WERE OBTAINED

Important applications, including the generation of antibodies and structural studies, require large amounts (several milligrams) of serine ADP-ribosylated peptides. In addition, the possibility of scaling up the reaction significantly facilitates the commercialisation of peptides, as a bulk of a serine ADP-ribosylated peptide can be aliquoted in tens or hundreds of vials that can be then sold separately. Under the following tested conditions, the inventors were able to produce ˜5 mg of pure serine mono-ADP-ribosylated peptide:

### Solvent: Water

Buffer: 50 mM Tris-HCl, pH=7.5;

### Salts: 50 mM NaCl and 1 mM MgCl₂;

### NAD⁺: 2 mM;

### PARP-1: 100 nM;

### HPF1: 1.5 μM;

Substrate peptide: 450 μM (Histone H3 (1-15) peptide)
Incubation time: 320 min

The reaction mix was incubated for 320 minutes at RT and stopped by adding 1 μM Olaparib. Afterwards, 1 μM PARG was added and the reaction mix and it was incubated for 60 minutes at RT. After checking the efficiency, yield and specificity of the reaction by using the novel strategy described in this application (see Example 4), the serine mono-ADP-ribosylated peptides were separated from the other constituents of the reaction mix by using reverse chromatography (C18 cartridge). Pure mono-Ser ADP-ribosylated H3 (1-15) were eluted with 30% Acetonitrile.

### EXAMPLE 4—REACTION ASSAY—A METHOD FOR CHECKING THE PURITY, YIELD AND SPECIFICITY OF SERINE ADP-RIBOSYLATION

In order to produce pure ADPr peptides a visualization methodology is necessary that allows not only the optimization of the reaction but also the assessment of the specificity, purity and yield of the modified peptide. The use of radioactive NAD⁺ as a substrate coupled with standard SDS-PAGE electrophoresis was the first and is still the primary means of visualizing in vitro ADP-ribosylated peptides (or proteins) in the art. In fact, it was the gel electrophoresis method that the inventors initially used to visualize Ser-ADPr peptides (and recombinant proteins) that was generated by reacting PARP-1, HPF1, and NAD⁺ in vitro (see FIG. 3 and Bonfiglio et al., 2017, loc. lit.).

However, with this radioactive NAD⁺ approach it is impossible to monitor the efficiency and the yield of the reaction generating Ser-ADPr peptides or proteins, since the unmodified substrate peptides or proteins are completely “invisible” with this visualization strategy. In addition standard gel electrophoresis conditions, such as those employed with the radioactive NAD⁺ approach, do not allow the spatial separation of ADPr peptides from the unmodified counterpart. Therefore, in order to be able to optimize the ADPr reaction, an unbiased and straightforward method for visualizing both the ADPr-modified and unmodified species was needed.

The novel visualization method is presented herein in FIGS. 1, 2A, C, D and E. The novelty is that a TBE-polyacrylamide gel, intended for electrophoresis of short nucleic acids, is adapted for the separation of peptides by switching the polarity of the electrophoresis runs. SDS-PAGE, which is the standard gel electrophoresis system for the separation of ADP-ribosylated species (FIG. 2B), does not allow the separation of an ADP-ribosylated peptide from its unmodified counterpart, as both peptides have similarly strong negative charges in the presence of SDS. Considering that ADP-ribose is a nucleotide (more precisely a dinucleotide) the inventors reasoned that an electrophoresis system, such as the TBE-polyacrylamide gel, that is capable of resolving one nucleotide difference in length of nucleic acid fragments would allow a clear separation between an ADP-ribosylated peptide and its unmodified counterpart. However, in a TBE gel the negatively-charged nucleic acids are separated by migrating toward the positively charged anode. The serine ADP-ribosylation substrate peptides, in contrast, have a net positive charge in the absence of SDS, even when modified by ADP-ribose, and, therefore, cannot be run on a TBE gel in its standard configuration. A simple solution to this practical problem is changing the polarity of the electrodes by reversing the jacks when connecting to the power supply. By this means, the positively-charged peptides can run into the commercial off-the-shelf TBE gel and be separated according to their charge. ADP-ribosylated peptides are less positively charged and have a higher mass compared to their unmodified counterparts and therefore migrate significantly more slowly, which allows a clear spatial separation between the bands of the modified and unmodified peptides. After the run, any simple peptide/protein staining strategy (e.g. Coomassie) can be used, and both species (unmodified and modified) are detected, as depicted in FIGS. 1, 2A, C, D and E.

### EXAMPLE 5—ADDITIONAL EXAMPLES FOR OBTAINING PURE SER-ADPR SUBSTRATES BY USING THE PRESENT INVENTION

To further demonstrate that the claimed ˜100% serine ADP-ribosylation method is applicable to virtually any peptide or protein with an amino acid sequence containing or comprising at least one serine, the inventors have applied the method of the present invention to different substrates as follows.
Substrate: 170 μM Histone H4 (1-19)-Biotin peptide
   Solvent: Water
   Buffer: 50 mM Tris-HCl, pH=7.5;
   Salts: 50 mM NaCl and 1 mM MgCl₂;
   NAD⁺: 2 mM (added every 120 min);
   PARP-1: 120 nM;
   HPF1: 1.5 μM;
   PARG: 1 μM
   Incubation time: 360 min
Substrate: 195 μM Histone H3 (21-44)-Biotin peptide
   Solvent: Water
   Buffer: 50 mM Tris-HCl, pH=7.5;
   Salts: 50 mM NaCl and 1 mM MgCl₂;
   NAD⁺: 2 mM;
   PARP-1: 120 nM;
   HPF1: 1 μM;
   Incubation time: 300 min
Substrate: 95 μM Histone H3 (1-21)-K9Me Biotin peptide
   Solvent: Water
   Buffer: 50 mM Tris-HCl, pH=7.5;
   Salts: 50 mM NaCl and 1 mM MgCl₂;
   NAD⁺: 2 mM (added every 120 min);
   PARP-1: 100 nM;
   HPF1: 1 μM;
   Incubation time: 360 min
Substrate: 95 μM Histone H3 (1-21)-K9Me2 Biotin peptide
   Solvent: Water
   Buffer: 50 mM Tris-HCl, pH=7.5;
   Salts: 50 mM NaCl and 1 mM MgCl₂;
   NAD⁺: 2 mM (added every 120 min);
   PARP-1: 100 nM;
   HPF1: 1 μM;
   Incubation time: 360 min
Substrate: 90 μM Histone H1.0 (94-112) peptide
   Solvent: Water
   Buffer: 50 mM Tris-HCl, pH=7.5;
   Salts: 50 mM NaCl and 1 mM MgCl₂;
   NAD⁺: 2 mM;
   PARP-1: 100 nM;
   HPF1: 0.5 μM;
   Incubation time: 120 min
Substrate: 100 μM Histone H1.2 (178-195) peptide
   Solvent: Water
   Buffer: 50 mM Tris-HCl, pH=7.5;
   Salts: 50 mM NaCl and 1 mM MgCl₂;
   NAD⁺: 2 mM;
   PARP-1: 100 nM;
   HPF1: 0.5 μM;
   Incubation time: 120 min
Substrate: 115 μM TMA16 (2-22) peptide
   Solvent: Water
   Buffer: 50 mM Tris-HCl, pH=7.5;
   Salts: 50 mM NaCl and 1 mM MgCl₂;
   NAD⁺: 2 mM;
   PARP-1: 100 nM;
   HPF1: 1.5 μM;
   Incubation time: 240 min

The results are shown in FIG. 4 and it is evident that ˜100% pure Ser-ADPr protein or peptide was obtained.

1. A method for the production of a serine ADP-ribosylated protein or peptide comprising preparing an aqueous buffered solution comprising 5 to 60 mM, preferably 10 to 60 mM of a buffer and having a pH between 5.0 and 9.0, preferably between 5.5 to 8.5, and most preferably between 6.1 to 8.3, said solution further comprising
(a) 0.2 to 2.5 mM NAD⁺,
(b) at least 50 nM, preferably 50 to 3000 nM PARP-1, PARP-2 or the PARP-1 variant E988Q,
(c) at least 100 nM, preferably 100 to 5000 nM HPF1,
(d) at least 10 μg/mL, preferably 10 μg/mL to 200 μg/mL sonicated DNA, said sonicated DNA preferably comprising DNA fragments of 10 to 330 bp, and
(e) up to 600 μM protein or peptide, said protein or peptide comprising at least one serine,
thereby generating a reaction mix, in which the protein or peptide becomes serine ADP-ribosylated.

2. The method of claim 1, wherein the solution further comprises at least 1 μM, preferably 1 to 10 μM PARG and/or the ADP-ribosylated protein or peptide is incubated with at least 1 μM, preferably 1 to 10 μM PARG, thereby obtaining a mono-ADP-ribosylated protein or peptide.

3. The method of claim 1, wherein the reaction is carried out at 15-35° C. and preferably at room temperature.

4. The method of claim 1, wherein the reaction is carried out for at least 90 min, preferably at least 120 min and more preferably at least 240 min.

5. The method of claim 1, wherein a fresh pool of 0.2 to 2.5 mM NAD⁺ is added to the reaction mix at least every 90 minutes, preferably at least every 60 minutes and more preferably at least every 45 minutes.

6. The method of claim 1, wherein the at least one serine is neighboured by at least one basic amino acid.

7. The method of claim 1, wherein the substrate contains a positively charged tail, a poly-arginine and/or lysine tail.

8. The method of claim 1, wherein the aqueous buffered solution further comprises
(f) 10 to 80 mM NaCl or KCl, and/or
(g) 0.5 to 2 mM MgCl₂,

9. The method of claim 1, wherein the buffer is
a Tris-HCl buffer and is preferably used at a final concentration of 40-60 mM,
a Hepes buffer and is preferably used at a final concentration of 40-60 mM, more preferably about 50 mM, or
a phosphate buffer and is preferably used at a final concentration of 5-20 mM, more preferably about 10 mM.

10. The method of claim 1, further comprising purifying the serine ADP-ribosylated protein or peptide from the reaction mix.

11. The method of claim 10, wherein the serine ADP-ribosylated protein or peptide is purified from the reaction mix by StageTip fractionation employing C8, C18, SCX, SAX or SDB-RPS chromatography media, cation or anion exchange chromatography, hydrophilic interaction chromatography, phosphopeptide enrichment, enrichment with a ADP-ribose-binding protein domain, boronate affinity chromatography, filtering the reaction with an ultrafiltration device, a spin column or a combination thereof.

12. The method of claim 1, wherein the efficiency of the serine ADP-ribosylation of the protein or peptide is checked by running the reaction product on a polyacrylamide gel with inverted polarity.

13. The method of claim 12, wherein the protein or peptide is stained, preferably with a Coomassie dye, silver staining or a reverse staining technique, such as imidazole reverse staining and zinc reverse staining, and is more preferably stained with Imperial™ protein stain.

14. The method of claim 1, wherein the method is carried out in vitro or ex vivo.

15. A kit for the production of a serine ADP-ribosylated protein or peptide comprising
(a) 5 to 60 mM, preferably 10 to 60 mM of a buffer having a pKₐ between 5.0 and 9.0, preferably between 5.5 and 8.5, and most preferably between 6.1 to 8.3,
(b) 0.2 to 2.5 mM NAD⁺,
(c) at least 50 nM, preferably 50 to 3000 nM PARP-1, PARP-2 or the PARP-1 variant,
(d) at least 100 nM, preferably 100 to 5000 nM HPF1,
(e) at least 10 μg/mL, preferably 10 μg/mL to 200 μg/mL sonicated DNA, said sonicated DNA preferably comprising DNA fragments of 10 to 330 bp,
(f) optionally at least 1 μM, preferably 1 to 10 μM PARG,
(g) optionally 10 to 80 mM NaCl or KCl, and
(h) optionally 0.5 to 2 mM MgCl₂,
in one or more container(s).
